(19) United States
(12) Patent Application Publication
(10) Pub. No.: US 2024/0190995 A1
(43) Pub. Date: Jun. 13, 2024

(54) SEPARATION MEDIA AND PURIFICATION METHODS FOR BLOOD ANTIBODIES USING THE SAME

(71) Applicant: Donaldson Company, Inc., Bloomington, MN (US)

(72) Inventors: Jinxiang Zhou, Greenville, SC (US); Graham Temples, Greenville, SC (US)

(21) Appl. No.: 18/383,464

(22) Filed: Oct. 24, 2023

Related U.S. Application Data
(60) Provisional application No. 63/419,207, filed on Oct. 25, 2022.

(51) Int. Cl. C07K 16/34; A61K 35/16; B01D 15/20; B01D 15/38; B01J 20/26; B01J 20/28; B01J 20/32

(52) U.S. Cl. CPC C07K 16/34; A61K 35/16; B01D 15/203; B01D 15/3809; B01J 20/264; B01J 20/28033; B01J 20/3219; B01J 20/3278

(57) ABSTRACT

Separation media that includes a support substrate and a plurality of separation ligands immobilized on the support substrate. The plurality of separation ligands include an affinity capable of recognizing and binding to blood type antigen recognizing domain. Methos of making the separation media and methods of using the separation media.

### CROSS-REFERENCE TO RELATED APPLICATION

This application claims the benefit of U.S. Provisional Patent Application No. 63/419,207, filed Oct. 25, 2022, which is incorporated herein by reference in its entirety.

### FIELD

The present disclosure relates to separation media and separation devices containing the same. The separation media of the present disclosure may be useful for isolation and/or concentration of biomolecules found in the blood. The separation media of the present disclosure may be used for separations in membrane chromatography. The present disclosure further relates to methods of making and using the separation media.

### INTRODUCTION

Blood components contain valuable biomolecules for fighting infection, clotting, and other biological processes. Blood products include whole blood and products isolated from whole blood including platelets, plasma, white blood cells, plasma fractionation products (e.g., antibodies isolated from whole blood), and cryoprecipitated antihemophilic factor. Patients receive blood product transfusions as therapeutic treatments for many diseases including hemophilia, Willebrand disease, burns, liver disease, disseminated intravascular coagulation, thrombocytopenia, sepsis, infections, cancer and cancer treatment side effect management, and many others. The demand for blood products is high. As such, further improvements to blood product isolation and purification are needed.

### SUMMARY

This disclosure describes, in one aspect, a separation media that includes a support substrate and a plurality of separation ligands immobilized on the support substrate. The plurality of separation ligands are of the formula SL:

-L-Z (SL)
where L is a linker and Z is a separation group. The separation group includes an affinity group. The affinity group includes a blood type antigen or a fragment thereof. In some embodiments, the formula SL is of formula SL1 or SL2:

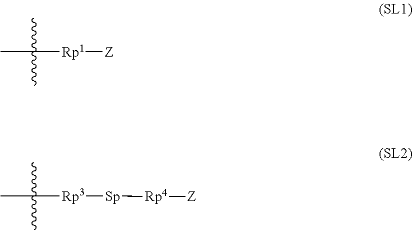

In formula SL1 and SL2 Rₚ¹, Rₚ³, and Rₚ⁴ each independently comprise the reaction product of any one of Rₚ^{A}, Rₚ^{B}, Rₚ^{C}, Rₚ^{D}, Rₚ^{E}, Rₚ^{F}, Rₚ^{G}, Rₚ^{H}, Rₚ^{I}, Rₚ^{J}, Rₚ^{K}, or an isomer thereof:

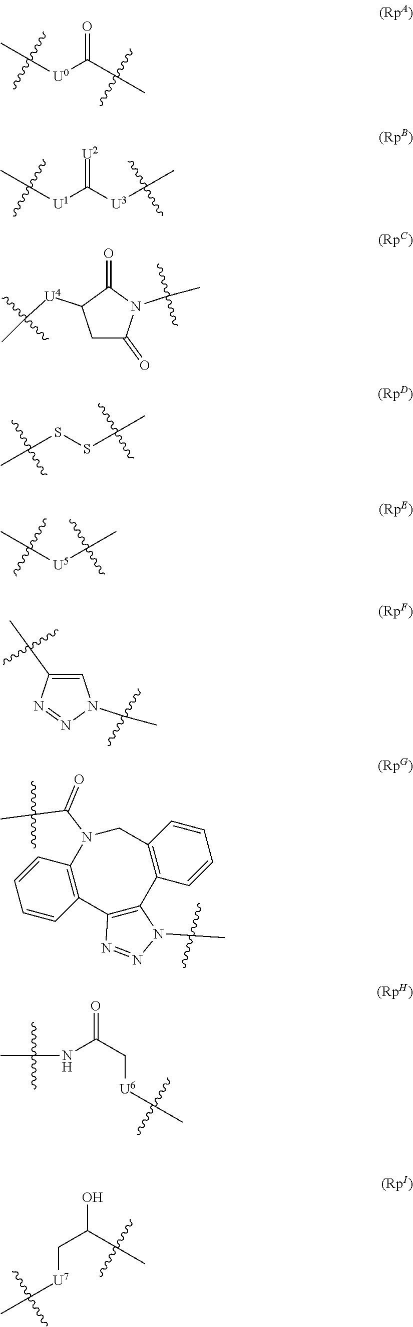

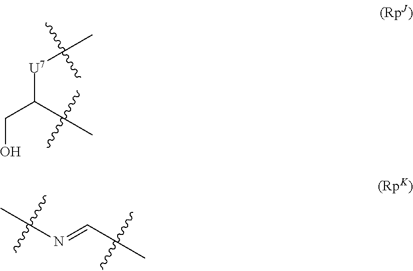

where U⁰, U¹, U², U³, U⁴, U⁵, U⁶, and U⁷ are each independently NH, N, O, or S and Sp is a spacer.

In another aspect, this disclosure describes a separation device that includes a housing and a separation media of the present disclosure disposed within the housing.

In another aspect, this disclosure describes a method for removing a target molecule from an isolation solution. The isolation solution includes an isolation solvent and the target molecule. The target molecule includes a blood antigen recognizing domain. The method includes contacting the isolation solution with the separation media or separation device of the present disclosure.

### BRIEF DESCRIPTION OF FIGURES

FIG. 1 is a schematic depiction of blood type antigens of the ABO blood group system.

FIG. 2A is a flow diagram of a first method for making the separation media of the present disclosure.

FIG. 2B is a flow diagram of a second method for making the separation media of the present disclosure.

FIG. 3A is a flow diagram of a third method for making the separation media of the present disclosure.

FIG. 3B is a flow diagram of a fourth method for making the separation media of the present disclosure.

FIG. 4A is a flow diagram of a fifth method for making the separation media of the present disclosure.

FIG. 4B is a flow diagram of a sixth method for making the separation media of the present disclosure.

FIG. 5A is a schematic of a separation media consistent with embodiments of the present disclosure.

FIG. 5B is a schematic representation of a separation device consistent with embodiments of the present disclosure.

FIG. 6 is a flow diagram of a method of using the separation media and/or separation devices of the present disclosure.

FIG. 7 is a first schematic synthetic strategy for the making of separation media consistent with the present disclosure. This strategy includes the deposition of a polymer onto the support substrate through the grafting on technique. This strategy also includes indirect immobilization of the separation ligands onto the support substrate and an amine assisted coupling method.

FIG. 8 is a second schematic synthetic strategy for the making of separation media consistent with the present disclosure. This strategy includes the deposition of a polymer onto the support substrate through the grafting from technique. This strategy also includes indirect immobilization of the separation ligands onto the support substrate and an amine assisted coupling method.

FIG. 9 is a third schematic synthetic strategy for the making of separation media consistent with the present disclosure. This strategy includes direct immobilization of the separation ligands onto the support substrate and an amine assisted conjugation method.

FIG. 10 is a fourth schematic synthetic strategy for the making of separation media consistent with the present disclosure. This strategy includes direct immobilization of the separation ligands onto the support substrate and an organic solvent assistance conjugation method.

### DEFINITIONS

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used here, the symbol

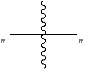

(referred to here as “a point of attachment bond”) denotes a bond that is a point of attachment between two chemical entities, or a chemical entity and a support substrate, one of which is depicted as being attached to the point of attachment bond and the other of which is not depicted. For example

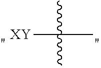

indicates that the chemical entity “XY” is bonded to another chemical entity or support substrate via the point of attachment bond.

The term “organic group” refers to a group that has carbon-hydrogen bonds. The group may also include heteroatoms such as O, S, N, or P. One or more heteroatoms may be catenated at any location in the organic group (e.g., ether, thioether, or amine). A heteroatom may be covalently bonded to a carbon atom through a double bond (e.g., ketone, imine). A heteroatom covalently bonded to a carbon atom may also be covalently bonded to another heteroatom (e.g., phosphodiester, sulfone). One or more functional groups may be included in an organic group, for example, alkane (branched, linear, or cyclic), alkene (branched or linear), alkyne (branched or linear), aromatic, amine (primary, secondar, tertiary, or quaternary), amino, amide, alcohol (primary, secondary, or tertiary), alkoxy, aldehyde, carboxylic acid, ether, ester, imine, phosphoester, phosphodiester, sulfone, sulfonamide, urea, thiourea, thioether, or any combination thereof, and ionized versions thereof. Generally, the organic group may be covalently bonded to compound. The point of attachment of the organic group to the compound may be described in several ways. For example, in some embodiments, the organic group may be described as the monovalent or radical of the respective functional group (e.g., alkyl for alkane, aryl for aromatic ring, aminyl for a primary or secondary amine). In some embodiments, where a general formula is shown with a covalent bond connecting the organic group to a compound, the organic group may be described as the common functional group. For example, if the organic group R is described relative to the formula CH₃CH₂CH₂—R, the organic group may be described, for example, as an aromatic ring.

The term “catenated” in the context of heteroatoms refers to a heteroatom (e.g., O, S, N, P) that replaces at least one carbon atom in a carbon chain. For example, ether groups contain one catenary oxygen atom with at least one carbon atom on each side of the catenary oxygen atom and polyether groups contain more than one catenary oxygen atom with carbon atoms on each side of the more than one catenary oxygen atoms.

The term “aryl” refers to a monovalent group that is aromatic. The aryl group may be carbocyclic or include one or more heteroatoms such as S, N, or O. Example aryl groups include, but are not limited to, phenyl, thiophenyl, furanyl, pyridinyl, pyrimidinyl, piperidinyl, and pyrrolyl.

The term “alkylene” or “alkanediyl refers to a divalent group that is a radical of an alkane and includes groups that are linear, branched, cyclic, bicyclic, or a combination thereof. Unless otherwise indicated, the alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. Examples of “alkylene” groups include methylene, ethylene, propylene, 1,4-butylene, 1,4-cyclohexylene, and 1,4-cyclohexyldimethylene.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Unless stated otherwise specifically in the specification, an alkyl group can be optionally substituted.

“Alkoxy” refers to the group —OR, where R is alkyl, alkenyl, alkynyl, cycloalkyl, or heterocycle as defined herein. Unless stated otherwise specifically in the specification, alkoxy can be optionally substituted.

The term “backbone” refers to the longest contiguous chain. One or more branches may be covalently bonded to the backbone.

The term “aromatic” refers to a cyclic, fully conjugated planar structure that obeys Wickes rules, that is the compound has 4n+2π electrons where n is a positive integer or zero. For example, benzene has 6 π electrons. Thus, 6=4n+2π. Solving for n gives 1. Therefore, benzene is an aromatic compound.

The term “kosmotrope” is generally used to denote a solute that increases the degree of ordered-ness of water by stabilizing water-water interactions. Kosmotropes may be ionic or non-ionic. In contrast, the term “chaotrope” is generally used to denote a solute that decreases the degree of ordered-ness of water by destabilizing water-water interactions. Chaotropes may be ionic or non-ionic.

The term “peptide” refers to a sequence of amino acid residues without regard to the length of the sequence. Therefore, the term “peptide” refers to any amino acid sequence having at least two amino acids and includes full-length proteins and, as the case may be, polyproteins.

The term “polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

The term “protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

In the description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

All scientific and technical terms used herein have meanings commonly used in the art unless otherwise specified. The definitions provided herein are to facilitate understanding of certain terms used frequently herein and are not meant to limit the scope of the present disclosure.

### DETAILED DESCRIPTION

The present disclosure provides separation media and separation devices containing the same. Specifically, the present disclosure provides separation media that may be used to remove antibodies that target blood type antigens from a mixture, such as from a blood product. To that end, the separation media of the present disclosure includes separation ligands. The separation ligands include a separating group that can be an affinity group, an assistance group, or a capping group. The affinity group includes a carbohydrate to which the target molecule (e.g., antibodies) can bind. Multiple layers of separation media of the present disclosure may be arranged in a stacked configuration to separate more than one target molecule, increase separation specificity, and/or increase efficiency. The separation media of the present disclosure may be used for separations in membrane chromatography.

Blood group systems and blood type are used to classify blood. A blood group system is a system in the human species where blood cell-surface antigens are controlled at a single gene locus or by two or more very closely linked homologous genes with little or no observable recombination between them. Blood type is the specific pattern of blood cell-surface antigens in an individual human including the blood groups from one or more blood group systems.

The ABO blood group system is the major blood group system used to classify blood. In the ABO system there are four major blood groups that are based on the presence or absence of two antigens displayed on the surface of red blood cells. Table 1 shows the four major blood groups (A, B, O, and AB), the antigens found on the surfaces of the red blood cells of the blood group, the antibodies in the blood of each blood group, and the compatibility of the blood groups. People with blood type A have A-type antigens and produce antibodies that bind to the B antigen (anti-B antibodies). People with blood type B have B-type antigens and produce antibodies that bind to the A antigen (anti-A antibodies). People with blood type AB have A-type and B-type antigens and produce no antibodies that would bind to the A or B antigens. People with blood type O have neither A-type nor B-type antigens and produce antibodies that bind to the A-type antigen and antibodies that bind to the B-type antigen. Due to the differences in antibodies, blood is not universal; that is, blood cannot be safely transferred between all humans. If a person receives blood from a donor having a blood type that is incompatible with the recipient's blood type, a severe life threatening reaction may occur.

TABLE 1
Major blood groups of the ABO blood group
system and characteristics of the same

| Blood | | Antibodies in | Blood group |
|---|---|---|---|
| Group | Antigens | blood | compatibility |
| A | A | anti-B | A and O |
| B | B | anti-A | B and O |
| O | none | anti-A and anti-B | O |
| AB | A and B | none | A, B, and O |

After the ABO blood group system, the Rh blood group system is the second major blood group system used to classify blood. There are more than 50 known Rh antigens with D, C, c, E and e being considered the most important. Individuals who are Rh positive display at least one

Rh antigen on the surface of their red blood cells. Individuals who are Rh negative do not display an Rh antigen on the surface of their red blood cells. Exposure of an individual having an Rh(D) negative blood type to blood from a donor having a Rh(D) positive blood type may result in the production of anti-D antibodies that bind to the Rh(D) positive blood cells causing a life-threatening reaction.

As is evident by the ABO and Rh blood group systems, blood group compatibility is of high importance for whole blood transfusion. Blood composition gets even more complex when the 41 other known blood group system recognized by the International Society of Blood Transfusion are considered. These systems include MNS, P, Lutheran, Kell, Lewis, Duffy, Kidd, Diego, Yt, XG, Scianna, Dombrock, Colton, Landsteiner-Wiener, Chido, Hh, XK, Gerbich, Cromer, Knops, Indian, Ok, Raph, JMH, li, globoside, GIL, Rh-associated glycoprotein, Forssman, Langereis, Junior, Vel, CD59, Augustine, KANNO, SID, CTL2, PEL, MAN, EMM, ABCC1, and Er. Between these systems there are over 600 blood group type antigens. Luckily, incompatibility between many of these blood group systems is not as common as incompatibility between ABO and Rh blood groups.

Blood products are increasingly being used as therapeutics. For example, in addition to the blood type antibodies, blood includes IgG, IgM, IgA, and IgD antibodies that have valuable therapeutic potential because they are a part of and stimulate immune responses to diseases and conditions. Blood products that include such antibodies can be transfused into patients to treat disease and various conditions. Unfortunately, due to blood type antibodies in the blood, patients receiving blood product transfusions are limited to receiving donations from donors having a compatible blood type.

Current blood product purification methods indiscriminately remove proteins and antibodies from blood products. As such, there is a need for separation media and separation methods for selectively removing blood type antibodies from blood products. As used here, a “blood product” is any therapeutic composition that has at least one component isolated from human blood. Blood products include platelets, plasma, white blood cells, plasma fractionation products (e.g., antibodies isolated from whole blood), cryoprecipitated antihemophilic factors, and any combinations thereof. As used here, a “blood type antibody” is an antibody that binds to a blood type antigen. The term “blood type antigen” refers to an antigen displayed on the surface of red blood cells that is a part of a blood group system.

Traditionally, downstream purification of biomolecules has been expensive, slow, and difficult to scale. Typical biomolecule purification trains include various steps such as centrifugation, filtering, and one or more chromatography separations using one or more types of chromatography columns (e.g., size exclusion columns and affinity chromatography columns). A typical chromatography column used in biomolecule purification may include a packed bed column with resin configured for size exclusion chromatography, reverse phase chromatography, or affinity chromatography. Resin based chromatography columns have been the gold standard employed to purify biologics for decades. However, column chromatography in large volumes may be very slow. Additionally, resin columns are known to require long residence times to perform adequately.

The present disclosure describes separation media that may be used for separation in membrane chromatography. In contrast to resin columns, membrane adsorbers perform well at short column residence times, potentially providing rapid separations for biologics. The present disclosure provides separation media that are suitable for separation, purification, and/or concentration of molecules that include a blood antigen recognizing domain. The term “blood type antigen recognizing domain” refers to a biomolecule, such as a protein, or a portion thereof, that is capable of recognizing a blood antigen. An example of a molecule that includes a blood type antigen recognizing domain is a blood type antibody.

Molecules of interest that may be separated using the separation media of the present disclosure are collectively referred to here as target molecules or targets. The target molecules of the present disclosure are molecules that include a blood type antigen recognizing domain, for example, a blood type antibody. The target molecule may be present in a solution, suspension, or dispersion. For simplicity, the liquid containing the target molecules is referred to here as an isolation solution. Also, for simplicity, a target may be referred to in the singular but it is understood that an isolation solution may include a plurality of target molecules of the same identity. The isolation solution may be a blood product or be a solution that includes a blood product. As such, the isolation solution may include other biomolecules. Some such biomolecules may be useful as therapeutics. The separation media may be used to remove or separate the target molecule from other biomolecules in the isolation solution. Once the target molecules are removed, the remaining biomolecules in the isolation solution may be used as a blood product or further processed. In some embodiments, the isolation solution includes plasma, platelets, white blood cells, plasma fractionation products (e.g., antibodies isolated from whole blood), cryoprecipitated antihemophilic factors, or combinations thereof. In some embodiments, the isolation solution includes a blood product that does not include red blood cells, white blood cells, or both. The isolation solution containing the target molecule may also include isolation solvents, such as water, organic solvents, or a combination thereof, and soluble components dissolved in the solvent. The separation media may be configured for use with organic solvents. The separation media may be configured to separate the target molecules from an isolation solution that includes organic solvents.

The separation media of the present disclosure include a plurality of separation ligands immobilized on a support substrate. The separation ligands include one or more separation groups. A separation group is a chemical group that facilitates the isolation of a target molecule from an isolation solution. Facilitation of separation may be in the form of a chemical group to which the target molecule binds; a chemical group that allows for increased density of the affinity group-target molecule interaction and/or increases the target molecule attraction to the support membrane; or a chemical group that blocks a reactive group from covalently modifying the target molecule during contact with the separation media; or a combination thereof. A separation group may be an affinity group, an assistance group, or a capping group. The separation media includes a plurality of separation ligands that include an affinity group. In addition to the plurality of separation ligands that include an affinity group, the separation media may include a plurality of separation ligands that include an assistance group; a plurality of separation ligands that include a capping group; or both.

A support substrate is the base material for the separation media. The support substrate provides a platform for which the separation ligands are immobilized. The support substrate includes at least one membrane. In some embodiments, the support substrate is the at least one membrane. In some embodiments, the support substrate includes two or more membranes arranged in a stacked configuration. In addition to the at least one membrane, the support substrate may include additional layers such as hydrogels, woven fibrous materials (i.e., a material made by the interlacing of multiple fibers), nonwoven fibrous materials (i.e., a material made from one or more fibers that are bound together through chemical, physical, heat, or mechanical treatment); or combinations thereof. Such additional layers may impart rigidity and structure to the support substrate. In some embodiments, the support substrate includes a functionalized material that is deposited on the surface of the at least one membrane. The functionalized material may provide reactive handles to which the separation ligands may be reacted to be immobilized to the support substrate. In embodiments where the separation media includes multiple layers, the layers may be laminated.

Any layer of the support substrate may be made of any suitable material. A suitable support substrate material is a material that is porous so as to allow the isolation solution to pass through the support substrate. In some embodiments, suitable support substrate material is a material that does not chemically alter the target molecule; that is, does not react with the target molecule to add, remove, or transform chemical groups on the target molecule. Additionally, in some embodiments, a suitable support substrate is a material that does not react with the target molecule, or other molecules in the isolation solution, to form a covalent bond which would permanently immobilize said molecule to the support substrate.

The support substrate includes at least one membrane. A membrane is understood as a sheet of material with a continuous pathway of polymeric material in all dimensions. The membrane may be made of any suitable support substrate material. Examples of suitable support substrate membrane materials include polyolefins; polyethersulfone; poly(tetrafluoroethylene); nylon; fiberglass; hydrogels; polyvinyl alcohol; natural polymers such as cellulose, cellulose ester, cellulose acetate, regenerated cellulose, cellulosic nanofiber, cellulose derivatives, agarose, chitosan; polyethylene; polyester; polysulfone; expanded polytetrafluoroethylene (ePTFE), polyvinylidene fluoride; polyamide (Nylon); polyacrylonitrile; polycarbonate; and any combination thereof.

In some embodiments, the support substrate itself is functionalized prior to immobilizing the separation ligands. Functionalization of the membrane may be done to install reactive handles (e.g., a support substrate reactive handle as discussed elsewhere herein) on the membrane. The reactive handles react with cooperative reactive handles on the separation ligands to form a covalent bond thereby immobilizing the separation ligands on the support substrate (as discussed elsewhere herein). Functionalization may be accomplished by plasma treatment, corona treatment, and the like.

In some embodiments, the support substrate includes a functionalized layer. In some embodiments, the functionalized layer is a membrane. A functionalized layer is a material disposed on the surface of a support substrate layer (e.g., disposed on the surface of the at least one membrane) and includes the support substrate reactive handles that may be used for separation ligand immobilization. A functionalized layer may be covalently attached to the support substrate; adhered to the support substrate through electrostatic forces, hydrogen-bonding, and/or Van der Waals forces; laminated to the support substrate; or simply contacting the support substrate. A functionalized layer may be deposited on the surface a support substrate (e.g., on the surface of the at least one membrane) using a variety of deposition techniques such as chemical vapor deposition, dip coating, spray coating, electrospinning, and the like.

In some embodiments, the functionalized layer is a polymer that is disposed onto the support surface using a grafting on or grafting from polymerization technique. The terms “grafting on,” “grafting onto,” and “grafted onto” refer to already formed polymer chains that adsorb or covalently attach to a surface (e.g., a support substrate surface). The terms “grafting from” or “grafted from” refer to a polymer chain that is imitated and grown from a surface (e.g., a support substrate surface). Any suitable polymer may be grafted on or grafted from a support substrate to from a functionalized layer. Suitable polymers are those that include a functional group that includes a reactive handle that allows for attachment of separation ligands to the support substrate. The reactive handle is not the polymerizable group, but instead is a group that remains intact following polymerization. Example polymers that include a reactive handle or a functional group that can be converted to a reactive handle (i.e., support substrate reactive handle) include carboxylic acids, amines, alcohols, epoxides, amides, azide, alkynes, and the like. Examples of monomers that can be used to form such polymers include vinyl alcohol, hydroxy functional acrylates (e.g., 2-hydroxyehtyl acrylate and 4-hydroxybutyl acrylate), hydroxy functional methacrylate (e.g., hydroxyethyl methacrylate), epoxy containing monomers, and hydroxy functional acrylamides (e.g., N-hydroxyethyl acrylamide). Examples of specific polymers that may be grafted on or grafted from a support substrate include polydopamine, poly(vinyl alcohol), poly(acrylic acid), poly(glycidyl methacrylate, and poly 2-hydroxyethyl acrylate (formed from 2-hydroxyethyl acrylate monomers). Graft on and graft from polymerization may be accomplished using suitable technique such addition polymerization (e.g., free radical polymerization such as atom transfer radical polymerization (ATRP) and reversible addition fragmentation chain transfer (RAFT) polymerization; anionic polymerization; and cationic polymerization) or condensation polymerization. Functionalized layer materials include poly(viny alcohol), polydopamine. In embodiments, where the polymer is grafted from the support substrate, an initiator is first coupled to the support substrate (e.g., through an OH group on the support substrate). Any suitable initiator may be used, for example, 2-bromo-2-methylpropionyl bromide (BiBB).

The membranes of the support substrate are porous and can have an average pore size, as measure by a capillary flow porometer, of 10 micrometer (μm) or less, 5 μm or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

In some embodiments, the support membrane includes cellulose such as regenerated cellulose, cellulose acetate, or cellulose ester. In some such embodiments, the support membrane has an average pore size 0.1 μm to 0.5 μm, 0.1 μm to 0.6 μm, 0.1 μm to 0.3 μm, or 0.4 μm to 0.6 μm.

The membrane may have a thickness of 500 μm or greater, 250 μm or greater, 100 μm or greater, 80 μm or greater, 50 μm or greater, or 30 μm or greater. The membrane may have a thickness of 2500 μm or less, 1000 μm or less, 500 μm or less, 250 μm or less, or 100 μm or less. The thickness of the membrane may be in a range of 30 μm to 500 μm, 50 μm to 500 μm, 80 μm to 500 μm, 100 μm to 500 μm, 250 μm to 500 μm, 30 μm to 250 μm, 50 μm to 250 μm, 80 μm to 250 μm, 100 μm to 2500 μm, 30 μm to 100 μm, 50 μm to 100 μm, or 80 μm to 100 μm.

In some embodiments, the support substrate includes multiple membranes stacked in a multilayer arrangement to increase capacity or selectivity of the separation media for a given application. The multilayer membrane configuration (i.e., only considering the membrane layers of a support substrate) maybe have a thickens of 10,000 micrometers (pm) or less, 7,500 μm or less, 5,000 μm or less, 4,000 μm or less, 3,000 μm or less, 2,500 μm or less, 2,000 μm or less, 1,000 μm or less, 750 μm or less, 500 μm or less, 400 μm or less, or 300 μm or less. The stacked arrangement of membranes may have a thickness ranging from 70 μm to 10,000 μm, 70 μm to 100 μm, 70 μm to 200 μm, 70 μm to 300 μm, 70 μm to 400 μm, 70 μm to 500 μm, 70 μm to 750 μm, 70 μm to 1,000 μm, 70 μm to 2,000 μm, 70 μm to 3,000 μm, 70 μm to 4,000 μm, 70 μm to 5,000 μm, 250 μm to 300 μm, 250 μm to 400 μm, 250 μm to 500 μm, 250 μm to 750 μm, 250 μm to 1,000 μm, 250 to 2,000 μm, 250 to 3,000 μm, 250 to 4,000 μm, 250 to 5,000 μm, 500 μm to 1,000 μm, 500 μm to 2,000 μm, 500 μm to 3,000 μm, 500 μm to 4,000 μm, or 500 μm to 5,000 μm in thickness.

In some embodiments, the membrane is a regenerated cellulose membrane having a pore size of between 0.2 μm and 5.0 μm, a thickness of between 70 μm and 2,000 μm. Such membranes may be in a stacked arrangement approximately 70 μm to 10,000 μm in thickness.

The support substrate may include or be a microfiltration membrane. Microfiltration membranes are typically created through a phase inversion process or an expansion process.

Typical materials used to prepare membranes include polyethersulfone (PES), nylon, polyvinylidene fluoride (PVDF), cellulose acetate, regenerated cellulose, polypropylene, and expanded polytetrafluoroethylene (ePTFE).

The separation media includes a plurality of separation ligands that include an affinity group. In addition to the plurality of separation ligands that include an affinity group, the separation media may include a plurality of separation ligands that include an assistance group; a plurality of ligands that include a capping group; or both.

An affinity group is a chemical group that is bound by the target molecule. Stated differently, an affinity group is a chemical group that binds the target molecule. The affinity group includes a blood type antigen or a portion thereof. The target molecules include a blood type antigen recognizing domain that can bind to the blood type antigen or a portion thereof thereby temporarily immobilizing the target molecule to the separation media. The target molecules may be a blood type antibody. In some embodiments, the target molecule is an anti-A antibody, an anti-B antibody, or both. The blood type antigen may be any blood type antigen. In some embodiments, the target molecule is an anti-Rh antibody.

Blood type antigens may be proteins, monosaccharides, disaccharides, or glycans (a polymer of three of more monosaccharides where each monosaccharide is linked to at least one adjacent saccharide through a glycosidic linkage). Some blood type antigens are proteins. Other blood type antigens are monosaccharide, disaccharides, or glycans that are conjugated to protein. Yet, other types of blood type antigens are monosaccharides, disaccharides, or glycans conjugated to lipids.

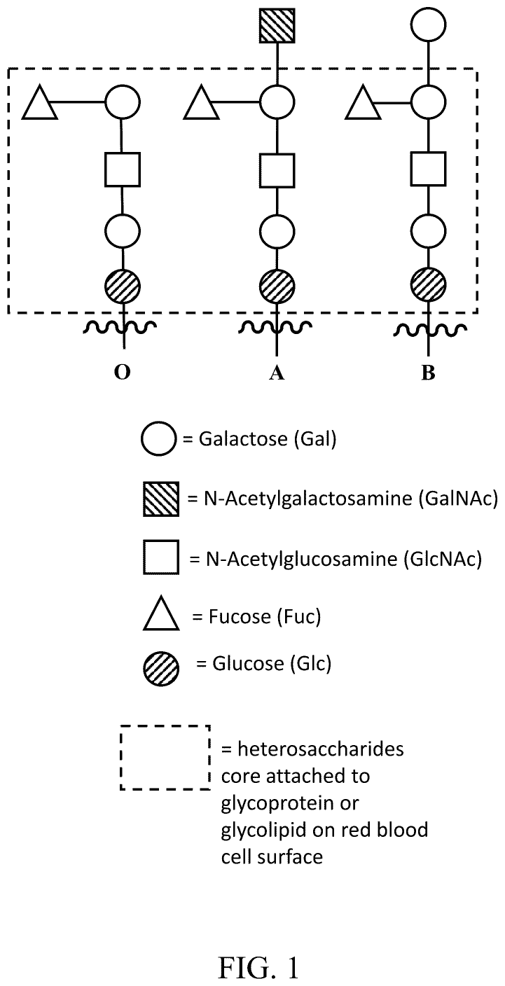

In some embodiments, the blood type antigen is a monosaccharide, a disaccharide, or a glycan. In some embodiments, the blood type antigen is the A-type antigen, the B-type antigen, or both. FIG. 1 shows the structure of the most prevalent A-type surface marker, the B-type surface marker, and the O-type surface marker. There are also known A sub-type antigens A1, A2, and A3. The glycans of the A-type, B-type, and O-type surface markers all include the same heterosaccharide core called the H antigen (dashed box). The A-type antigen and the B-type antigen differ from the O surface markers (the H antigen) in that they include an additional terminal monosaccharide. When used in reference to a blood type antigen, the term “terminal” refers to the end of the disaccharide or glycan that is free and not attached to a lipid or a protein in the context of a red blood cell. The A-type antigen and the B-type antigen differ in the identity of the terminal saccharide. The A-type antigen has a terminal N-acetylglucosamine saccharide residue, and the B-type antigen has a terminal galactose residue.

In some embodiments, the affinity group is a blood type antigen glycan or a portion thereof. In some embodiments, the affinity group includes the entire blood type antigen glycan. In other embodiments, the affinity group includes only a portion of the blood type antigen glycan. In some such embodiments, the affinity group includes 1, 2, 3, 4, 5, 6, 7, 8, 9, or 10 of the saccharides at the terminal end of the glycan. In some such embodiments, the affinity group includes the 3 or 6 saccharides at the terminal end of the glycan. In some embodiments, the affinity group includes the H antigen. In some embodiments, the affinity group includes the H antigen and a terminal GalNAc. In some embodiments, the affinity group includes the H antigen and a terminal GlcNAc. In some embodiments, the affinity group includes the same composition and bond configuration as the natural blood type antigen glycan. In some embodiments, the affinity group includes the last 3 saccharides of the terminal end of the blood type antigen.

In some embodiments, the affinity group includes the A-type antigen or a portion thereof. In such embodiments, the target molecule may be an anti-A antibody. In some embodiments, the affinity group includes the entire A-type antigen. In other embodiments, the affinity group includes the last 1, 2, 3, 4, 5, or 6 saccharides of the terminal end of the glycan of the A-type antigen. In some embodiments, the affinity group includes Gal, Fuc, and GalNAc arranged in the same order and bond linkages as in the A-type antigen. In some embodiments, the affinity group includes the H antigen and a terminal GalNAc.

In some embodiments, the affinity group includes the B-type antigen or a portion thereof. In such embodiments, the target molecule may be an anti-B antibody. In some embodiments, the affinity group includes the entire B-type antigen. In other embodiments, the affinity group includes the last 1, 2, 3, 4, 5 or 6 saccharides of the terminal end of the glycan of the B-type antigen. In some embodiments, the affinity group includes the H antigen and a terminal GlcNAc. In some embodiments, the affinity group includes Gal, Fuc, and GlcNAc arranged in the same order and bond linkages as in the A-type antigen. In some embodiments, the affinity group includes the H antigen and a terminal GlcNAc.

In some embodiments, the affinity group includes an Rh-type antigen or a portion thereof. The Rh antigen may be any Rh antigen or a portion thereof such as an Rh(D) antigen, Rh(C) antigen, Rh(E) antigen, Rh(c) antigen, or Rh(e) antigen.

In some embodiments, the separation media includes a first plurality of separation ligands that include a separation group having an affinity group that includes a first blood type antigen or a portion thereof and a second plurality of separation ligands that include a separation group that has an affinity group that includes a second blood type antigen or a portion thereof. Such a separation media may be used to remove two or more target molecules from an isolation solution. For example, in embodiments where the first blood type antigen is an A-type antigen and the second blood type antigen is a B-type antigen, the separation media may be used to remove both anti-A and anti-B antibodies from an isolation solution, for example, a blood product.

The ratio of the first blood type antigen to the second blood type antigen may be designed to reflect the theorized amounts of each target molecule in the isolation solution. For example, in some embodiments, the isolation solution includes a blood product that has been pooled from two or more donors of a population. The ratio of the first blood type antigen (first affinity group) to the second blood type antigen (second affinity group) may be based on the amount of the population that has each ABO blood groups. For example, a blood product has been pooled from a population where one third or the population has the A blood group, one third of the population has the B blood group, and one third of the population has the O blood group. The A blood group has anti-B antibodies, the B blood group has anti-A antibodies, and the O blood group has both anti-A and anti-B antibodies. If the O blood group has the same amount of anti-A and anti-B antibodies, then the ratio of anti-A to anti-B antibodies in the pooled blood product is 1 to 1. For such a population of donors, the separation media may be designed to have a 1 to 1 ratio of the separation ligands having an A-type antigen or fragment thereof to the separation ligands having a B-type antigen or fragment thereof.

In another embodiment, two or more layers of separation media may be arranged in a stacked configuration in order to remove two or more target molecules from an isolation solution. For example, a first separation media having separation ligands having a first blood type antigen affinity group or fragment thereof may be stacked with a second separation media having separation ligands having a second blood type antigen affinity group or fragment thereof. In some such embodiments, the first blood type antigen affinity group is a A-type antigen or fragment thereof and the second blood type antigen affinity group is a B-type antigen or fragment thereof. In other such embodiments, the second blood type antigen affinity group is a A-type antigen or fragment thereof and the first blood type antigen affinity group is a B-type antigen or fragment thereof. Additional separation media having the same or different affinity groups may be further included in the stacked configuration.

In some embodiments, the separation media includes a plurality of separation ligands that include a separation group that is an assistance group. An assistance group is a chemical moiety that facilitates the binding of the target molecule to the affinity group; binds the target molecule through electrostatic interactions and/or hydrophobic interactions; or both. In some embodiments, the assistance group may allow for a high density of target molecules to bind to separation ligands that include an affinity group. In some embodiments, the assistance group may aid in attracting the target molecule to the support substrate such as to allow for the target molecule to be in proximity to a separation group that includes an affinity group. For example, the assistance group may be ionizable or possesses a formal charge which may be opposite the charge of the target molecule. In such cases, the oppositely charged assistance group may attract the target molecule to the support substrate which may allow the target molecule to bind to the affinity group.

In some embodiments, the assistance group functions as a cation or anion exchange chromatography ligand. Anion exchange ligands have a positively charged functional group that targets negatively charged target molecules through electrostatic interactions. The anion exchange ligand may possess a formal positive charge, or the positive charge can be induced through the pH of the solution that the anion exchange ligand is exposed to. Cation exchange ligands have a positively charged functional groups that target negatively charged target molecules through electrostatic interactions. The anion exchange ligand may possess a formal negative charge, or the negative charge can be induced through the pH of the solution that the anion exchange ligand is exposed to.

In some embodiments, the assistance group possesses a positive formal charge or is ionizable under certain pH conditions to have a positive charge. Such assistance groups may be beneficial when the target molecule has a negative formal charge. Examples of such assistance ligands include primary, secondary, tertiary, and quaternary amines. Suitable amines may be diamines, triamines, and polyamines.

Examples of primary amines include methylene diamine, ethylene diamine, propylene diamine, butylenediamine (putrescine), pentylamine, or any aliphatic diamine with 1-18 carbons between the terminal amines, covalently attached via one of the amines. Such ligands can be made from polyamines such as ethylene diamine, diethylenetriamine, triethylenetetramine covalently attached via one of the amines.

Examples of secondary amines can include any of the aforementioned primary amines immobilized to the substrate, substituted with an additional R-group as described above. In cases in which diamines are used, secondary amines may also be formed by covalent interaction with the substrate coupling both amines to the substrate. Ligands containing secondary amines with the structure of the ligand may also be immobilized such as linear polyethyleneimine, spermidine, or spermine. Furthermore, groups containing a non-terminal primary amine (e.g., 3-aminopentane) may also be conjugated to the substrate to result in a secondary amine.

Examples of suitable tertiary amines include N,N-dimethylethylenediamine; N,N-dimethylpropylenediamine; N,N-diethylpropylenediamine; or any aliphatic diamine with aliphatic carbon group substitution on one or both amines ranging from one to six carbons, with and a linker having 2-18 carbons between the terminal amines.

Examples of quaternary amines include any of the aforementioned primary amines that have undergone a quaternarization reaction resulting in a permanent positive charge. Such reactions can be performed with alkyl groups such as methyl iodide or aryl groups such as benzyl iodide. Quaternary amines can further include any of the aforementioned tertiary amines that have undergone a quaternarization reaction resulting in a permanent positive charge. Such reactions can be described by the Menshutkin reaction which uses an alkyl halide to form a quaternary ammonium salt from a reaction with a tertiary amine. Such reactions can be performed with alkyl containing groups of varying length such as butyl bromide or aryl groups such as benzyl chloride or combinations therein. Additionally, compounds containing quaternary amines can be immobilized directly.

In other embodiments, the assistance group possesses a negative formal charge or is ionizable under certain pH conditions to have a negative charge. Such assistance group may be beneficial when the target molecule has a positive formal charge. The difference in charge of target molecule and the assistance molecule may allow for an electrostatic interaction between the target molecule and the assistance group thereby allowing the target molecule to be proximate to the support surface and the affinity groups which may increase the probability of the target molecule of binding to an affinity group.

In some embodiments, the assistance group is such that it is able to induce hydrophobic interactions with the target molecules. Hydrophobic interactions exploit the differences in hydrophobicity of between the target molecules and possible impurities. In one embodiment, such ligands include aliphatic chains with three carbons or longer (common used lengths include butyl, pentyl, hexyl, heptyl, octyl, nonyl, decyl, undecyl, and dodecyl), benzyl, phenyl, phenol, pyridine, boronic acid groups, branched polymers such as polypropylene glycol, and sulfur-containing thiophilic ligands such as propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, 2-phenylethanethiol, and the like, and combinations thereof

In some embodiments, separation ligands that include an assistance group can be directly incorporated into a functionalized layer of a support substrate through polymerization of a monomer that includes an assistance group.

In some embodiments, the separation media includes a plurality of separation ligands that includes a separation group that is a capping group. A capping group is a chemical moiety that prevents reactive groups of the support substrate from reacting with the target molecule or any other molecule in the isolation solution. A capping group may be employed to block support substrate reactive handles that have not reacted with other separation ligands. . A capping group may be used to cap the end of a polymer chain. Capping groups may be any chemical group that is non-reactive towards the target molecule or other molecules the isolation solution.

In some embodiments, a separation ligand immobilized on a support substrate has the formula (SLim):

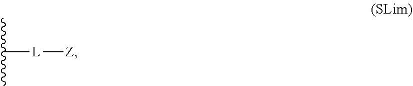

where L is a linker, Z is a separation group, and the vertical black line is the support substrate.

Each separation ligand of the plurality of separation ligands has the formula SL:

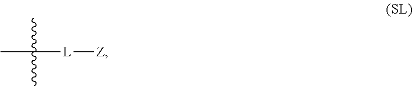

where L is a linker and Z is a separation group. L separates the support substrate from Z. The separation group may include an affinity group, capping group, or assistance group. The affinity group may be any affinity group as disclosed herein. The capping group may be any capping group as disclosed herein. The assistance group may be any capping group as disclosed herein.

Separation ligands of multiple chemical compositions may be immobilized to a single support substrate. For example, a support substrate may include a first portion of a separation ligands of formula SL and a second portion of separation ligands of formula SL. In some embodiments, the first portion and the second portion of separation ligands include the same affinity group but have different linkers (L). In other embodiments, the first portion and the second portion of the separation ligands may have the same linker but have different separation group.

In some embodiments, L is of formula L1 such that the separation ligand of formula SL is of formula SL1:

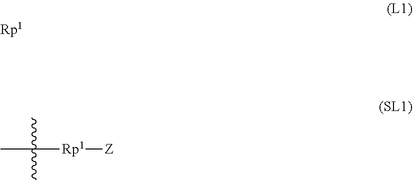

Rp¹ is a reaction product, and Z is the separation group. A reaction product is the chemical group resulting from the reaction of two cooperative functional handles (as discussed herein). In a separation ligand of formula SL1, the linker is the reaction product. The reaction product Rp¹ links the support substrate and the separation group (Z). A covalent bond from Rp¹ to the support substrate (not shown) is the point of covalent attachment of the linker (L1) to the support substrate. A covalent bond from Rp¹ to the separation group (Z) is the point of covalent attachment of the linker (L1) to the separation group (Z). Rp¹ may be any reaction product as disclosed herein.

The reaction product (Rp¹) may be the reaction product between any two cooperative reactive handles (as described herein). Examples of reaction products include amides, ureas, thioureas, carbamates, carbonates, esters, thioethers, ethers, and triazoles. In some embodiments, a reaction product (e.g., such as RP') is Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof:

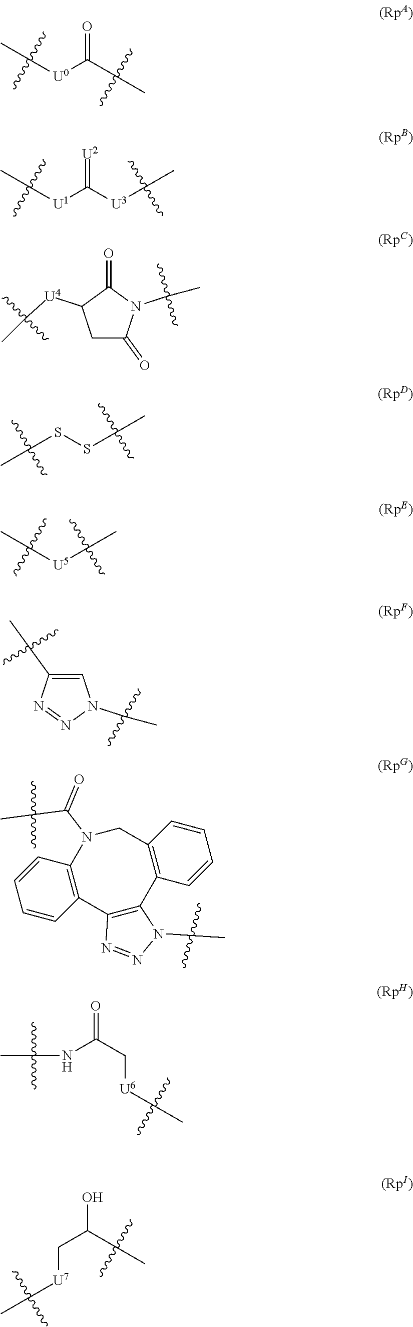

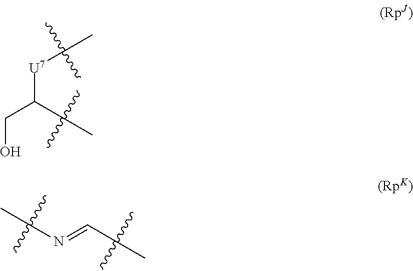

U⁰, U⁴, U⁵, U⁶, and U⁷ (found in Rp^{A}, Rp^{C}, Rp^{E}, Rp^{H}, Rp^{I}, and Rp^{J} respectively) are each independently NH, N, O, or S. For Rp^{B} each U¹, U², and U³ are independently NH, N, O, or S. The reaction products have two connection points, each of which may be covalently linked to the support substrate or any component of a separation ligand. For separation ligands of formula SL1 one connection point the reaction product Rp¹ is linked to the separation group while the other connection point of the reaction product Rp¹ is linked to the support substrate.

In some embodiments where the separation ligand is of formula SL1, Rp¹ is Rp^{A} where U⁰ is NH. In some such embodiments, the amide nitrogen (U⁰) of Rp^{A} is covalently linked to the separation group. In other such embodiments, the amide nitrogen of Rp^{A} is covalently linked to the support substrate.

In some embodiments where the separation ligand is of formula SL1, Rp¹ is Rp^{A} where U⁰ is O. In some such embodiments, the ester oxygen (U⁰) of Rp^{A} is covalently linked to the separation group. In other such embodiments, the ester oxygen of Rp^{A} is covalently linked to the support substrate.

The identity of a reaction product (e.g., Rp¹) depends at least in part on the type of conjugation chemistry used to form the reaction product. In a conjugation reaction, each component being linked together includes a reactive handle, such that the reactive handles are cooperative reactive handles. Components that include a reactive handle for conjugation reactions are termed precursor compounds or precursors. A precursor compound includes the component and a reactive handle covalently linked to the component. Cooperative handles or cooperative reactive handles are two or more reactive handles that when exposed to each other under favorable reaction conditions a conjugation reaction occurs to form a reaction product between the reactive handles. Components that have been conjugated through a conjugation reaction may be referred to as a conjugate. For example, component A and component B are to be conjugated through a conjugation reaction. The component A precursor includes a reactive handle X. The component B precursor includes a reactive handle Y. X and Y are cooperative. A conjugation reaction between the component A precursor and the component B precursor results in the formation of an A-B conjugate that includes the reaction product between X and Y. It is understood that the notation of a conjugate is from the perspective of the conjugated components, not the precursors of those components (i.e., A-B conjugate not A precursor-B precursor conjugate). This is because upon completion of the conjugation reaction, the precursor components are no longer precursors. In the case of a component precursor that includes two independently reactive handles, one of which has been reacted with a different component precursor to form a conjugate, the conjugate notation is still from the perspective of the conjugated components, not the precursor components, with the understanding that the conjugate includes the unreacted second reactive handle. For example, a component D precursor includes a first reactive handle J and a second reactive handle Z. The component B precursor has the reactive handle Y. J and Y are cooperative handles. A conjugation reaction between the component A precursor and the component B precursor results in the formation of an A-B conjugate that includes the reaction product between J and Y. The A-B conjugate also includes the unreacted second reactive handle Z.

Any pair of cooperative reactive handles may be used to forma reaction product of the present disclosure. Examples of cooperative handles include an activated ester and an amine; an amine and an NETS-ester; a hydroxyl and an NHS-ester; a hydroxyl and an epoxide; an acyl chloride and an amine; and acyl chloride and a alcohol; an amine and an epoxide; a thiol and an epoxide; a thiol and a maleimide; a disulfide and a thiol; an azide and an alkyne (azide and a linear alkyne in the presence of Cu(I); an azide and a cyclic alkyne such as cyclooctyne, difluorinated cyclooctyne, dibenzocyclooctyne, TMTH-SulfoxImine, biarylazacyclooctynone, or bicyclo[6.1.0]nonyne); an amine and an isocyanate; an amine and an isothiocyanate, a amine and a benzoyl fluoride; a thiol and a iodoacetamide; a thiol and a bromoacetamide; a disulfide and 2-thiopyridine; a thiol and 3-arylpropiolonitirle; a phenol and a diazonium salt; a phenol and 4-phenyl-1,2,4-triazoline-3,5-dione; a phenol and aldehyde, and a aniline; a hydroxyl and sodium periodate; a thiol and an iodoacetamide; an amine and a pyridoxal phosphate; an azide and a functionalized triphenyl phosphine; a tetrazine and a strained alkene; and the like.

Examples of individual reactive handles that may be used to form the separation media of the present disclosure include Rh^{A} (hydroxyl), Rh^{B} (thiol), Rh^{C} (amine), Rh^{D} (activated ester), Rh^{E} (azide), Rh^{F} (alkyne), Rh^{G} (NHS-ester), Rh^{H} (maleimide), Rh¹ (where X is a Cl, Br, or I leaving group attached to carbon that can undergo nucleophilic substitution; e.g., a bromoacetamide or iodoacetamide), Rh^{J} (cyclooctyne), Rh^{K} (isocyanate), Rh^{L} (isothiocyanate), Rh^{M} (where X is a Cl, Br, or I leaving group attached to carbon that can undergo nucleophilic substitution), Rh^{N} (an epoxide), Rh^{O} (an acyl chloride), Rh^{P} (aldehyde), and isomers thereof. Chemical structures of Rh^{A}-Rh^{P} are depicted below.

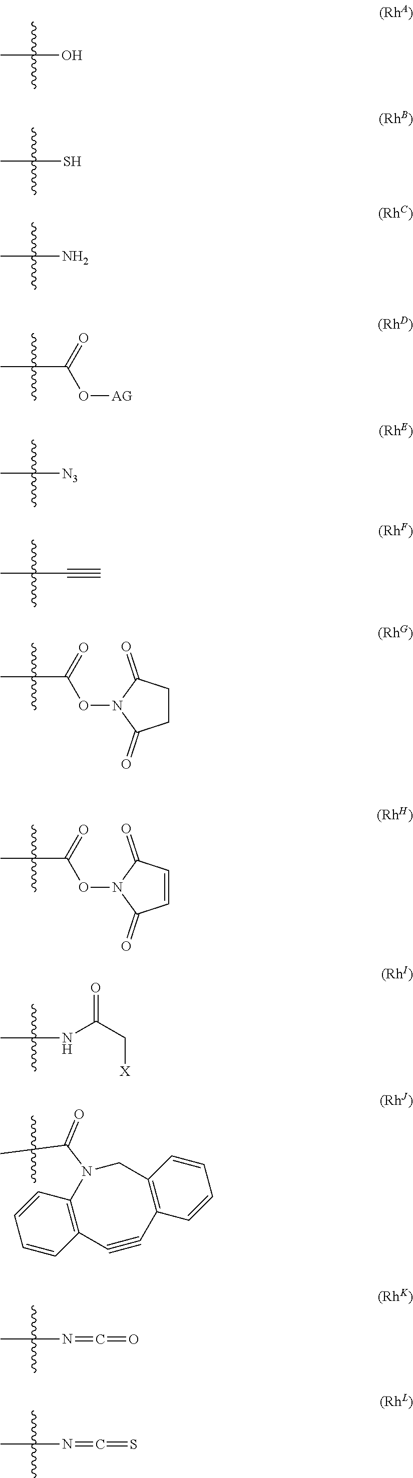

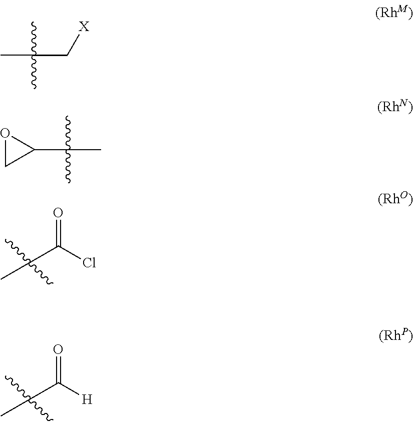

where X in Rh^{M} and Rh¹ may be -chloro, -bromo, or -iodo.

Rh^{D} is an activated ester where AG is an activating group. An activated ester is an ester that is reactive with an activated ester cooperative reaction handle (e.g., an amide) in a conjugation reaction. Activated esters may be denoted as the type of activated ester or by the activating group. Examples of activating groups include O-acylisoureas, benzotriazoles (with a bond between the ester oxygen and one nitrogen of the triazole), and pentafluorophenyl. In some embodiments, Rh^{D} may be an activated ester of a carboxylic acid. In such embodiments, the activated ester is formed through the reaction of a carboxylic acid with one or more reagents that install the activating group. For example, a carboxylic acid may be converted into an activated ester having a O-acylisoureas activating group by treating the carboxylic acid with various carbodiimide reagents (e.g., N,N′-dicyclohexylcarbodiimide, dirnethylaminopropyl)carbodiimide, diisopropylcarbodiimide (DIC)) under favorable reaction conditions. A carboxylic acid may be converted into an activated ester having a benzotriazole activating group by treating the carboxylic acid with various carbodiimide reagents followed by treatment with hydroxybenzotriazole (HOBT) or by treating the carboxylic acid with various benzotriazole containing compounds (e.g., O-(Benzotriazol-1-yl)-N,N,N′,N′-tetramethyluronium hexafluorophosphate (HBTU); O-(Benzotriazol-1-yl)- N,N,N′,N′-tetramethyluronium tetrafluoroborate (TBTU); 2-(1H-benzotriazol-1-yl)-1,1,3,3-tetramethyluroniurn hexafluorophosphate (HBTU); Benzotriazol-1-yloxy)tris(dimethylamino)phosphonium hexafluorophosphate (BOP); (Benzotriazol-1-yloxy)tripyrrolidinophosphonium hexafluorophosphate (PyBOP); and O-(7-Azabenzotriazol-1-yl)- N,N,N′,N′-tetramethyluroniunt tetrafluoroborate (TATU) under favorable reaction conditions. Other reagents are available for making activated esters from carboxylic acids including brontotripyrrolidinophosphonium hexafluorophosphate (PyBrOP); O-(N-succinimidyl)-1,1,3,3-tetramethyl-uronium tetrafluoroborate (TSTU); O-(5-Norbornene-2,3-dicarboxirnido)-N,N,N′,N′-tetrarnethyluronium tetrafluoroborate (TNTU); O-(1,2-Dihydro-2-oxo-1-pyridyl-N,N,N′,N′-tetratriethyluronium tetrafluoroborate (TPTU); and 3-(diethylphosphoryloxy)-1,2,3-benzotriazin-4(3H)-one (DEPBT); carbonyldiimidazole. In sonic embodiments, the activated ester may be created in situ from a carboxylic acid and not isolated prior to a conjugation reaction.

Rh^{O} is an acyl chloride. Acyl chlorides may be prepared from carboxylic acids, for example, using thionyl chloride. Acyl chlorides may not be stable and as such, may be prepared in situ and not isolated prior to a conjugation reaction.

Reactive handles Rh^{A}, Rh^{B}, Rh^{C}, Rh^{D}, Rh^{E}, Rh^{F}, Rh^{G}, Rh^{H}, Rh^{I}, Rh^{J}, Rh^{K}, Rh^{L}, Rh^{M}, Rh^{N}, Rh^{O}, and Rh^{P} include various pairs of cooperative handles that can from the reaction products of Rh^{A}, Rp^{B}, Rp^{C}, Rp^{D}, Rp^{E}, Rp^{F}, Rp^{G}, Rp^{H}, Rp^{I}, Rp^{J}, and Rp^{K}. For example, under favorable reaction conditions, a conjugation reaction between Rh^{A} and Rh^{D} forms Rp^{A} where U⁰ is O. Under favorable reaction conditions, a conjugation reaction between Rp^{D} and Rh^{C} forms Rp^{A} where U⁰ is NH. Under favorable reaction conditions, a conjugation reaction between Rh^{C} and Rh^{G} forms Rp^{A} where U⁰ is NH. Under favorable reaction conditions, a conjugation reaction between Rh^{B} and Rh^{H} forms Rp^{C} where U⁴ is S. Under favorable reaction conditions, a conjugation reaction between two Rh^{B} forms Rp^{D}. Under favorable reaction conditions, a conjugation reaction between Rh^{C} and Rh^{I} forms Rp^{H} where U⁶ is NH. Under favorable reaction conditions, a conjugation reaction between Rh^{B} and Rh^{I} forms Rp^{H} where U⁶ is S. Under favorable reaction conditions, a conjugation reaction between Rh^{M} and Rh^{B} forms Rp^{E} where U⁵ is S. Under favorable reaction conditions, a conjugation reaction between Rh^{M} and Rh^{C} forms Rp^{E} where U⁵ is NH. Under favorable reaction conditions, a conjugation reaction between Rh^{K} and Rh^{C} forms Rp^{B} where U¹ and U³ are NH and U² is O. Under favorable reaction conditions, a conjugation reaction between Rh^{L} and Rh^{C} forms Rp^{B} where U¹ and U³ are NH and U² is S. Under favorable reaction conditions, a conjugation reaction between Rh^{F} and Rh^{E} forms Rp^{F}. Under favorable reaction conditions, a conjugation reaction between Rh^{J} and Rh^{E} forms Rp^{G}. Under favorable reaction conditions, a conjugation reaction between Rh^{N} and Rh^{A} forms Rp¹ or Rp^{J} where U⁷ is O. Under favorable reaction conditions, a conjugation reaction between Rh^{N} and Rh^{B} forms Rp¹ or Rp^{J} where U⁷ is S. Under favorable reaction conditions, a conjugation reaction between Rh^{N} and Rh^{C} forms Rp^{I} or Rp^{J} where U⁷ is N. Under favorable reaction conditions, a conjugation reaction between Rh^{O} and Rh^{A} forms Rp^{A} where U⁰ is O. Under favorable reaction conditions, a conjugation reaction between Rh^{O} and Rh^{B} forms Rp^{A} where U⁰ is NH. Under favorable reaction conditions, a conjugation reaction between Rh^{P} and Rh^{C} forms Rp^{K}.

Conjugation reactions between cooperative handles may be done under favorable reaction conditions. Favorable reaction conditions are conditions that facilitate a reaction, increase the yield of a reaction, minimize unwanted biproducts of a reaction, and/or increase the rate of a reaction. Example reaction conditions include reaction temperature, reaction atmosphere composition, reaction solvent, the presence of a catalyst, the presence of a base, the presence of an acid, and combinations thereof. Favorable reaction conditions for conjugation reactions are known.

Cooperative handles may be chosen such that the conjugation reaction is an orthogonal conjugation reaction. Orthogonal conjugation reactions are reactions where the chemistry is selective such that only two cooperative handles react to form a reaction product even when additional reactive handles or pairs of cooperative reactive handles may be present. Orthogonal conjugation reactions may be useful because they allow for multiple selective conjugation reactions to take place in series or in parallel. Orthogonality of two or more conjugation reactions may be achieved by choosing reactive handles that are only reactive with their cooperative counterpart in the presence of other cooperative reactive handle pairs. Orthogonality of two or more conjugation reactions may also be achieved by using reactive handles that are reactive with multiple cooperative counterparts, but the reactivity can be influenced through the reaction conditions such that only a specific pair of cooperative handles will react in the given set of reaction conditions.

To form a separation ligand of formula SL1, conjugation reaction precursor compounds are employed, each precursor compound having a reactive handle that is cooperative with the reactive handle of a different precursor compound. In some embodiments, a separation ligand of formula SL1 is formed through the conjugation of a separation group precursor of formula Pre-Z(1) and a support substrate precursor of formula Pre-M(1) by way of synthetic scheme S1. The support substrate precursor includes a support substrate (thick black vertical line) and a support substrate reactive handle Rh¹ that is covalently attached to the support substrate. The separation group precursor includes the separation group (Z) of formula SL1 and a separation group reactive handle R². Rh¹ and Rh² are cooperative reactive handles and may be any pair of cooperative handles as disclosed herein. In scheme S1, the support substrate reactive handle (Rh¹) is reacted with the separation group reactive handle (Rh¹) to from a reaction product (Rp¹) thereby forming a separation ligand of formula SL1.

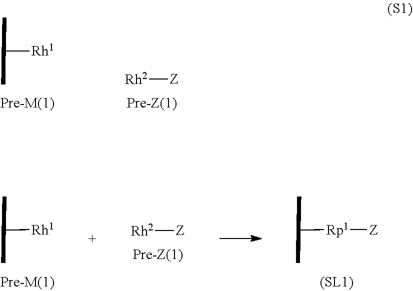

In some embodiments the material of the support substrate does not include a reactive handle that is cooperative with the separation group reactive handle (Rh²). In such embodiments, scheme S1 may further include installing the support substrate reactive handle Rh¹. The support substrate reactive handle Rh¹ may be installed through treatment of the support substrate to form the Rh¹. In such embodiments, a chemical functionality already present on the support substrate is transformed into the support substrate reactive handle. For example, the support substrate may be exposed to an oxidizing or reducing reagent (or conditions). The support substrate reactive handle Rh¹ may be installed through the installation of a functionalized layer. In such embodiments, the functionalized layer is considered a part of the support substrate. In such embodiments, the reactive handle of functionalized layer is the support substrate reactive handle. Examples of materials suitable for a functionalized layer are discussed herein.

In some embodiments, the linker of formula L1 of formula SL1 is derived from an amino acid or a peptide. In some embodiments, the linker of formula L1 is designed to mimic N-glycosidic linkages of glycans to an asparagine residue or peptides and/or proteins found in nature. In some such embodiments, the separation ligand may be of formula X, XI, XIII, or XIII.

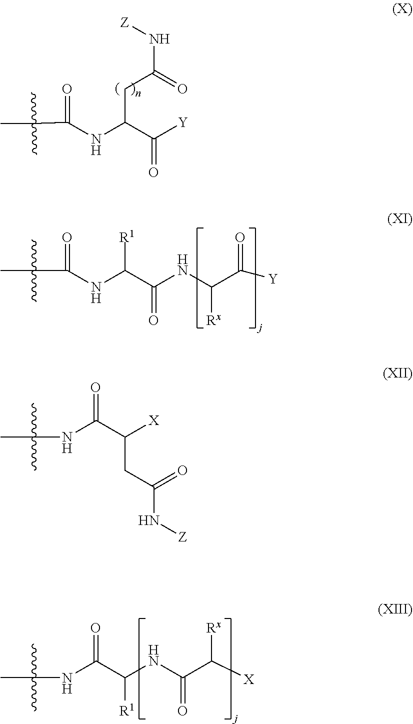

where Z is the separation group; R¹ is an amino acid side chain or a protected amino acid side chain; X is NH₂ or PG_{N} where PG_{N} is an amine protecting group; Y is OH or a PG_{C(O)OH} where PG_{C(O)OH} is a carboxylic acid protecting group; each R^{X} is an amino acid side chain, a protected amino acid side chain, or

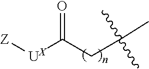

where at least one R^{X} is

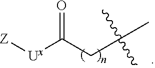

Each U^{X} is NH or O. j is 1, 2, 3, 4, 5, 6, 7, 8, 9, or 10. Formula X, XI, XII, and XIII all have a first reaction product Rp¹ that is Rp^{A} where U⁰ is NH.

R¹ and each R^{X} may be any amino acid side chain. An amino acid side chain is the chemical group extending from the alpha carbon of the amino acid. In some embodiments, R¹ and each R^{X} may independently be the amino acid side chain of arginine, histidine, lysine, aspartic acid, glutamic acid, serine, threonine, asparagine, glutamine, cysteine, selenocysteine, alanine, valine, isoleucine, leucine, methionine, phenylalanine, tyrosine, tryptophane, or an unnatural amino acid. The amino acid side chain may be a protected amino acid side chain; that is, the amino acid side chain may include a protecting group that masks a reactive group. For example, the amine of the side chain of lysine may be protected with an amine protecting group (e.g., tert-butyloxycarbonyl; allyloxycarbonyl; and benzyloxycarbonyl). The carboxylic acid of the side chain of aspartic acid and glutamic acid may be protected with a carboxylic acid protecting group (e.g., methyl ester; tert-butyl ester; 2,4-dimethyoxybenzyl ester; 9-fluorenylmethyl ester; and benzyl ester). The guanidinium of the side chain of arginine may be protected with a guanidinium protecting group (e.g., 2,2,4,6,7,-pentamethyl-2,3-dihydrobenzofuran-5-sulfonyl). The amide of the side chain of asparagine and glutamine may be protected with an amide protecting group (e.g., 9-xanthenyl). The thiol of the side chain of cysteine may include a thiol protecting group (e.g., trityl, p-methylbenzyl and acetamidomethyl). The side chain of serine or threonine may include an alcohol protecting group (e.g., tert-butyldimethylsilyl; allyl; and o-nitrobenzyl).

In some embodiments, X is an amine protecting group (PG_{N}). In some embodiments, Y is a carboxylic acid protecting group (PG_{C(O)OH}). PG_{N} and PG_{C(O)OH} may be any amine and carboxylic acid protecting group, respectively, as described herein.

In some embodiments, a separation ligand of formula X, XI, XII, or XIII can be formed from a single conjugation reaction; that is, by way of synthetic scheme 1 (S1). More specifically, separation ligand of formula X can be formed by way of synthetic scheme S1(a); a separation ligand of formula XII can be formed by way of synthetic scheme S1(b); a separation ligand of formula XII can be formed by way of synthetic scheme S1(c); and a separation ligand of formula XIII can be formed by way of synthetic scheme S1(d) where the formula for the support substrate precursor (Pre-M(1a) and Pre-M(11))) and the separation group precursor (Pre-Z(1a), Pre-Z(1b), Pre-Z(1c), and Pre-Z(1d)) are shown for each respective synthetic scheme.

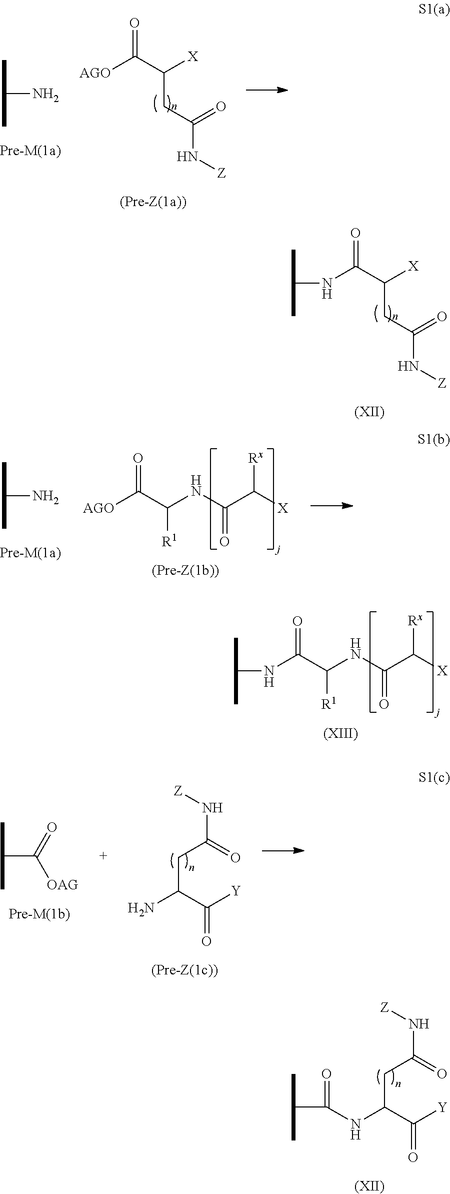

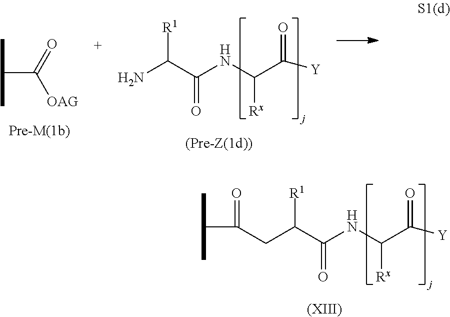

In scheme S1(a) and S1(b), the support substrate precursor (Pre-M(1a)) includes an amine reactive handle (i.e., the support substrate reactive handle). The counterpart separation group precursors include an activated ester reactive handle (i.e., the separation group reactive handle). The amine reactive handle is reacted with the activated ester reactive handle to form an amide reaction product (i.e., Rp¹ of scheme S1).

In scheme S1(c) and S1(d), the support substrate precursor (Pre-M(1b)) includes an activated ester reactive handle (i.e., the support substrate reactive handle). The counterpart separation group precursors include an amine reactive handle (i.e., the separation group reactive handle). The amine reactive handle is reacted with the activated ester reactive handle to form an amide reaction product (i.e., Rp¹ of scheme S1).

The conjugation reactions of S1(a), S1(b), S1(c), and S1(d) may be conducted similar to solid phase peptide coupling chemistry. In some such embodiments, the scheme further includes preparing the activated ester by reacting the carboxylic acid with an activating reagent to form the activated ester. The activated ester may be prepared from a carboxylic acid using reagents as described elsewhere herein. The activated ester is then reacted with the amine to form an amide bond (e.g., a peptide bond). Following the conjugation, any amino acid, carboxylic acid, or amine protecting groups can be deprotected if desired.

In some embodiments, the amine reactive handle of the separation group precursor (e.g., Pre-Z(1c) or Pre-Z(1d)) may be protected by an amine protecting group. In such embodiments, the synthetic scheme may first include deprotecting the protected amine to expose the amine reactive handle (i.e., the separation group reactive handle).

In some embodiments, the carboxylic acid from which the activated ester reactive handle is derived of the separation group precursor (e.g., Pre-Z(1a) or Pre-Z(1b)) may be protected by a carboxylic acid protecting group. In such embodiments, the synthetic scheme may first include deprotecting the protected carboxylic acid to expose the carboxylic acid. The scheme may further include reacting a carboxylic acid with a carboxylic acid activating reagent to form the activated ester reactive handle (i.e., the separation group reactive handle).

In some embodiments, the linker (L) is of formula L2 such that SM is of formula SL2; that is.

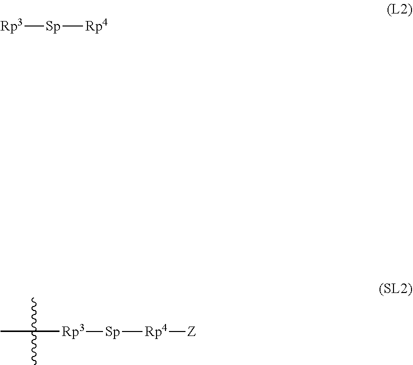

In formulas L2 and SL2, Sp is a spacer, Rp³ is a first reaction product, and Rp⁴ is a second reaction product. In formula SL2, Z is the separation group. The linker of formula L2 includes Sp, Rp³ and Rp⁴. In a separation ligand of formula SL2, a covalent bond from Rp³ to the support substrate is the point of covalent attachment of the linker (L2) to the support substrate. A covalent bond from Rp⁴ to the separation group (Z) is the point of covalent attachment of the linker (L2) to the separation group (Z). Rp³ and Rp⁴ may be any reaction product as described herein.

In some embodiments where the separation ligand is of formula SL2, Rp³, Rp⁴, or both are Rp^{A} where U⁰ is NH. In some such embodiments, the amide nitrogen (U⁰) of Rp^{A} is covalently linked to the support substrate or covalently linked to the separation group. In other such embodiments, the amide nitrogen of Rp^{A} is covalently linked to the spacer.

In some embodiments where the separation ligand is of formula SL2, Rp³, Rp^{A}, or both are Rp^{A} where U⁰ is 0. In some such embodiments, the ester oxygen (U⁰) of Rp^{A} is covalently linked to the support substrate or covalently linked to the separation group. In other such embodiments, the ester oxygen of Rp^{A} is covalently linked to the spacer.

The spacer (Sp) of the linker (L1) may be of any length and/or chemical composition that does not completely inhibit the formation of the first reaction product and the second reaction product. The spacer (Sp) may be of any length and/or chemical composition that does not completely inhibit the ability of the affinity group to bind to its intended target.

The spacer (Sp) includes a divalent organic group. The divalent organic group includes a backbone. The “backbone” is the longest contiguous chain of atoms within the spacer (Sp). In some embodiments, the backbone is a carbon-based backbone. A backbone that is carbon-based is a backbone that has a greater number of carbon atoms than heteroatoms in the backbone. The backbone may include one or more substitutions extending from the backbone and/or one or more functional groups catenated within the backbone.

In some embodiments, the backbone is an alkanediyl (divalent group that is a radical of an alkane) or an alkenediyl (divalent group that is a radical of an alkene). The alkanediyl or alkenediyl may have a backbone chain length of C1 to C18, C1 to C10, C1 to C6, C1 to C4, C1 to C3, or C2 to C4. An alkenediyl may have one or more double bonds. The one or more double bonds may be located at any point along the backbone.

In some embodiments, the backbone includes one or more catenated functional groups. Catenated functional groups have at least one atom that is a part of the backbone; that is, at least one atom of the functional group lies within the backbone chain. The at least one atom of the functional group that is a part of the backbone can be a carbon or a heteroatom. For example, in some embodiments, the backbone includes a catenated ketone where the carbon atom of the carbonyl of the ketone is a part of the backbone. In other embodiments, the backbone includes a catenated amide. In some such embodiments, the nitrogen of the catenated amide is a part of the backbone and the carbon of the carbonyl is not a part of the backbone. In other such embodiments, the nitrogen and the carbonyl carbon of the amide are a part of the backbone. Example catenated functional groups include, ethers; thioether; esters (where the ester oxygen atom is a part of the backbone, or where the ester oxygen and the carbonyl carbon are a part of the backbone); thioesters (where the thioester sulfur atom is a part of the backbone, or where the thioester sulfur atom and the carbonyl carbon are a part of the backbone); amides (where the amide nitrogen is a part of the backbone, or where the amide nitrogen and the carbonyl carbon are a part of the backbone); ureas (where one of the urea nitrogens is a part of the backbone, or where both of the urea nitrogens and the carbonyl carbon are a part of the backbone); carbamates (where the carbamate oxygen is a part of the backbone; the carbamate nitrogen is a part of the backbone; or the carbamate oxygen, the carbamate nitrogen, and the carbonyl carbon are a part of the backbone); thioureas (where one of the urea nitrogens is a part of the backbone, or where both of the urea nitrogens and the carbonyl carbon are a part of the backbone); secondary and tertiary amines; aromatic rings (where at least two atoms of the aromatic ring are a part of the backbone); and combinations thereof.

In some embodiments the spacer includes a catenated ether (i.e., a catenated oxygen atom). In some such embodiments, the backbone includes a polyethylene glycol chain of 1, 2, 3, 4, 5, 6, 7, 8, 9, or 10 —OCH₂CH₂— repeat units.

In some embodiments, the spacer includes a catenated ketone. In some such embodiments, the spacer is of the formula —(CO)—. In some embodiments, the spacer includes a C1 alkenediyl backbone and a catenated carbonyl where the C1 is the carbonyl carbon of the catenated ketone.

In some embodiments where the separation ligand is of formula SL2, Rp³ and Rp⁴ are both Rp^{E} where each U⁵ is independently O, NH, or S. In some embodiments, the U⁵ of Rp³ is O and the U⁵ of Rp⁴ is O. In some embodiments, the U⁵ of Rp³ is NH and the U⁵ of Rp⁴ is NH. In some embodiments, the U⁵ of Rp³ is O and the U⁵ of Rp⁴ is NH. In some embodiments, the U⁵ of Rp³ is NH and the U⁵ of Rp⁴ are O.

In some embodiments where Rp³ and Rp⁴ are both Rp^{E}, Sp may be —C(O)—. In some such embodiments, L2 may be described as Rp^{b}. In some embodiments were L2 is Rp^{B}, U² is O. In some embodiments were L2 is Rp^{B}, U¹ is O. In some embodiments were L2 is Rp^{B}, U³ is O. In some embodiments were L2 is Rp^{B}, U¹ is NH. In some embodiments were L2 is Rp^{B}, U³ is NH. In some embodiments were L2 is Rp^{B}, U¹ is O, U² is O, and U³ is NH. In some embodiments were L2 is Rp^{B}, U¹ is NH, U² is O, and U³ is O.

In some embodiments where the separation ligand is of formula SL2, Rp³ is Rp^{E} and Rp⁴is Rp^{I} or Rp^{J} where U 5 and U⁷ are each independently O, NH, or S. In some embodiments U⁵ is NH and U⁷ is NH. In some embodiments U⁵ is O and U⁷ is O. In some embodiments U⁵ is NH and U⁷ is 0. In some embodiments U⁵ is O and U⁷ is NH.

In some embodiments where Rp³ and Rp⁴ are both Rp^{E}, Sp may be —(CH₂)ₙ— where n is 0, 1, 2, 3, 4, 5, 6, 7, 8, 9 or 10. In some such embodiments, L2 is of the formula

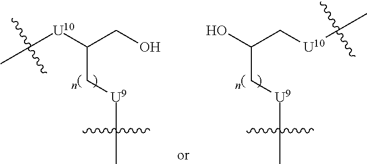

where U⁹ and U¹⁰ are each independently O, NH, or S and n is 0, 1, 2, 3, 4, 5, 6, 7, 8, 9, or 10. U⁹ may be U⁵ from Rp¹ and U¹⁰ may be U⁷ from Rp^{J}. In some embodiments U⁹ is NH and U¹⁰ is NH. In some embodiments U⁹ is O and U¹⁰ is O. In some embodiments U⁹ is NH and U¹⁰ is O. In some embodiments U⁹ is O and U¹⁰ is NH.

In some embodiments, a separation ligand of formula SL is of formula

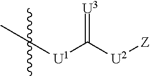

where U¹, U², and U³ are each independently O, NH, or S and Z is a separation group. For example, in some embodiments, a separation ligand of formula SL is

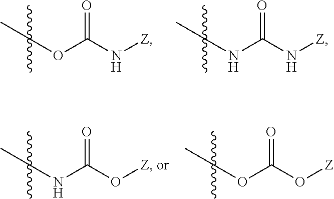

where Z is a separation group. In some embodiments, Z is an affinity group.

In some embodiments, a separation ligand of formula SL is of formula

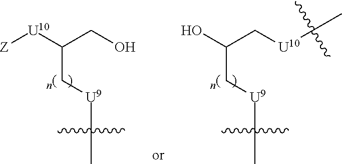

U⁹ and U¹⁰ are each independently O, NH, or S where Z is a separation group. In some embodiments, SL is

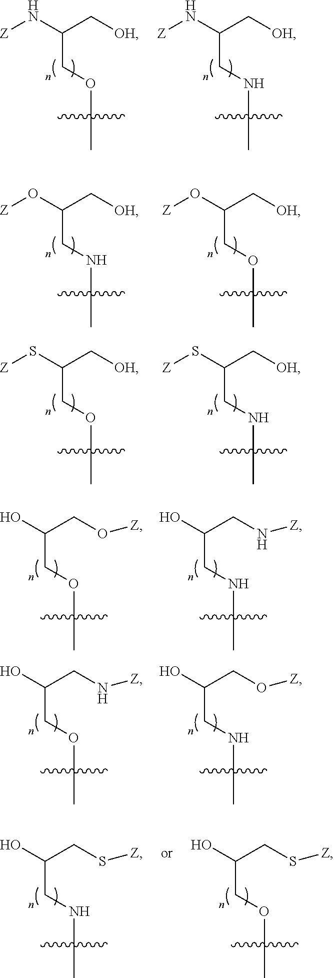

where Z is a separation group and n is 0, 1, 2, 3, 4, 5, 6, 7, 8, 9, or 10. In some embodiments Z is an affinity group.

To form a separation ligand of formula SL2, a series of conjugation reaction precursor compounds are employed, each precursor compound having a reactive handle that is cooperative with the reactive handle of a different precursor compound. In some embodiments, a separation media of formula SL2 is formed through the conjugation of a linker precursor of formula Pre-L, an affinity group precursor of formula Pre-Z(2), and a support substrate precursor of formula Pre-M(2). The linker precursor (Pre-L) includes the spacer Sp of the separation media of formula SL2, a first linker reactive handle Rh³, and a second linker reactive handle Rh⁴. The support substrate precursor (Pre-M(2)) includes a support substrate (thick vertical black line) and a support substrate reactive handle Rh⁵. The separation group precursor (Pre-Z(2)) includes the separation group Z of formula SL2 and a separation group reactive handle Rh⁶.

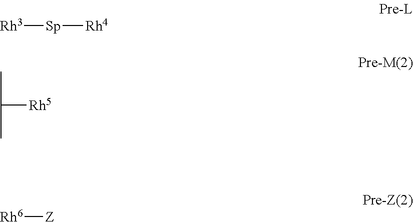

Rh³ and Rh⁵ are a pair of cooperative reactive handles. Rh⁴ and Rh⁶ are a pair of cooperative reactive handles. Rh¹ of the linker precursor reacts with Rh⁵ of the support substrate precursor in a conjugation reaction to from a reaction product (i.e., Rp³ of formula SL2). Rh¹ of the linker precursor reacts with Rh⁶ of the separation group precursor in a conjugation reaction to from a reaction product (i.e., Rp⁴ of formula SL2).

Because the linker precursor includes two reactive handles, the linker precursor is a bifunctional linker. In some embodiments, the linker precursor may be a multifunctional linker precursor that has three or more reactive handles. At least one of the reactive handles is configured to react with the support substrate precursor. The additional reactive handles may be configured to react with a cooperative reactive handle on one or more separation groups. Examples, of bifunctional and multifunctional linker precursors include, epichlorohydrin, diglycidyl ether, triglycidyl ether, tetraglycidyl ether, triazine, poly triazine, poly acrylic (e.g., the COOH groups can be made into activated ester reactive handles), succinic acid (e.g., the COOH groups can be made into activated ester reactive handles), and N′N′-disuccinimidyl carbonate (DSC).

In some embodiments, a separation ligand of formula SL2 may be formed through two conjugation reactions. The reactions may be conducted in any order or simultaneously. For example, in some embodiments, a separation ligand of formula SL2 is formed by way of synthetic scheme 2 (S2).

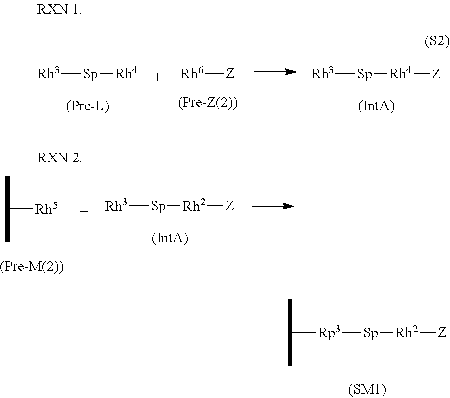

In a first conjugation reaction of scheme S2 (RXN1), the separation group reactive handle (Rh⁶) is reacted with a first linker reactive handle (Rh⁴) in a first conjugation reaction to from a first reaction product Rp⁴ thereby resulting in intermediate A (IntA). Intermediate A is a linker-separation group conjugate that includes the first reaction product (Rp⁴) and the second linker reactive handle (Rh³). IntA may be isolated or taken forward to the second conjugation reaction without isolation. In a second conjugation reaction of S2 (RXN 2) the second linker reactive handle (Rh³) of IntA is reacted with the support substrate reactive handle (Rh⁵) to form a second reaction product (Rp³), thereby forming a separation ligand of formula SL2.

In some embodiments, a separation ligand of formula SL2 is formed by way of synthetic scheme 3 (S3).

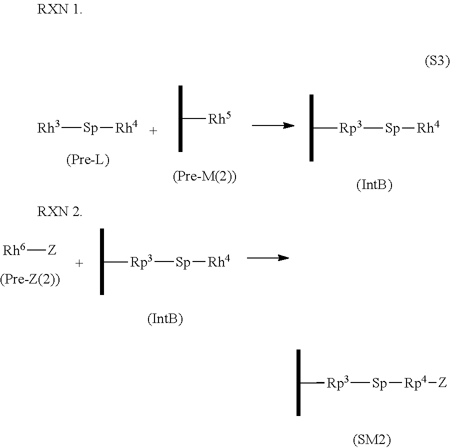

In a first conjugation reaction of scheme S3 (RXN1), the support substrate reactive handle (Rh⁵) is reacted with a first linker reactive handle (Rh³) in a first conjugation reaction to from a first reaction product (Rp³) thereby resulting in intermediate B (IntB). Intermediate B is a linker-support substrate conjugate that includes the first reaction product (Rp³) and the second linker reactive handle (Rh⁴). IntB may be isolated or taken forward to the second conjugation reaction without isolation. In a second conjugation reaction of S3 (RXN 2) the second linker reactive handle (Rh⁴) of IntB is reacted with the separation group reactive handle (Rh⁶) to form a second reaction product (Rp⁴), thereby forming a separation ligand of formula SL2.

Synthetic scheme S4 and synthetic scheme S5 are examples of forming a separation ligand of formula SL2 through scheme S3 using the bifunctional linker (Pre-L) N,N′-disuccinimidyl carbonate (S3) or epichlorohydrin (S4). In both S4 and S5, R¹⁰ can be OH, NH₂, or SH and R¹¹ can be O, NH, or S depending on the identity of R¹⁰.

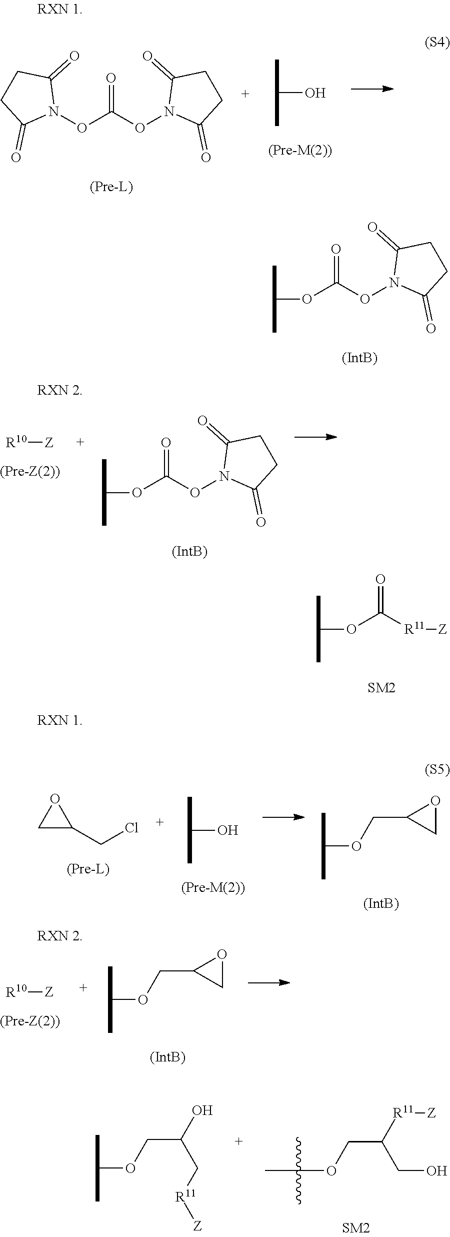

It is noted that separation ligands of formula X, XI, XII, and XIII include both a fist reaction product and a second reaction product (Rp^{A} where U⁰ is NH). As such, a separation ligand of any one of formula X, XI, XII, XIII, may be synthesized according to scheme S2 or S3 given the appropriate precursor compounds.

The present disclosure provides methods of making the separation media of the present disclosure. The separation media may be made methods described in PCT application number PCT/US2019/065805 (WO2020123714A1, Zhou), which is incorporated by reference in its entirety.

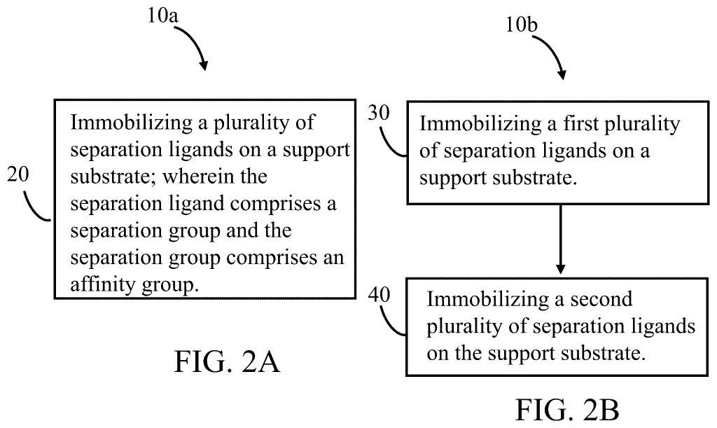

FIG. 2A is a flow diagram depicting a general method 10a for making a separation media of the present disclosure. The general method 10a includes immobilizing a plurality of separation ligands on a support substrate (step 20). Each separation ligand includes a separation group and a linker. The separation group includes the affinity ligand. Each separation ligand may be of formula SM, SM1, SM2, X, XI, XII, or XIII. Each separation ligand can be immobilized according to any relevant synthetic scheme described herein (e.g., S1, S1(a), S1(b), S1(c), S1(d), S2, S3, S4, or S5).

In some embodiments, the separation media includes a first plurality of separation ligands immobilized on the support substrate and a second plurality of separation ligands immobilized on the support substrate. FIG. 2B is a flow diagram depicting a general method 10b for making a separation media of the present disclosure that includes at least two pluralities of separation ligands. Each separation ligand of the first plurality of separation ligands and the second plurality of separation ligands includes a separation group and a linker. Each plurality of separation ligands immobilized on a support substrate may be of formula SLim. Each separation ligand of the first plurality of separation ligands and the second plurality of separation ligands may be of formula SM, SM1, SM2, X, XI, XII, or XIII. Each separation ligand of the first plurality of separation ligands and the second plurality of separation ligands can be immobilized according to any relevant synthetic scheme described herein (e.g., S1, S1(a), S1(b), S1(c), S1(d), S2, or S3, S4, or S5).

In some embodiments of method 10b, the first plurality of separation ligands includes an assistance group, and the second plurality of separation ligands includes an affinity group. Without wishing to be bound by theory, it is thought that the assistance groups of the first plurality of separation ligands can interact with (e.g., via electrostatics and/or hydrophobic or hydrophilic interactions) with the affinity group of the separation group precursor used to form the second plurality of separation ligands. Through these interactions, the second separation group precursors may concentrate on the surface of the support substrate thereby increasing conjugation reaction efficiency (e.g., speed and/or yield). An increase in reaction efficiency may allow a lower concentration of the second plurality of the second separation group precursors to be used in the reaction step than would be needed to achieve the same reaction yield and/or surface coverage without the use of assistance groups. In some embodiments, the assistance group includes an amine. In such embodiments where separation ligands that include an amine assistance group are immobilized prior to immobilization of separation ligands containing affinity groups, the method is amine assisted.

The method 10b includes immobilizing the first plurality of separation ligands on a support substrate (step 30). The method 10b further includes immobilizing the second plurality of separation ligands on the support substrate (step 40).

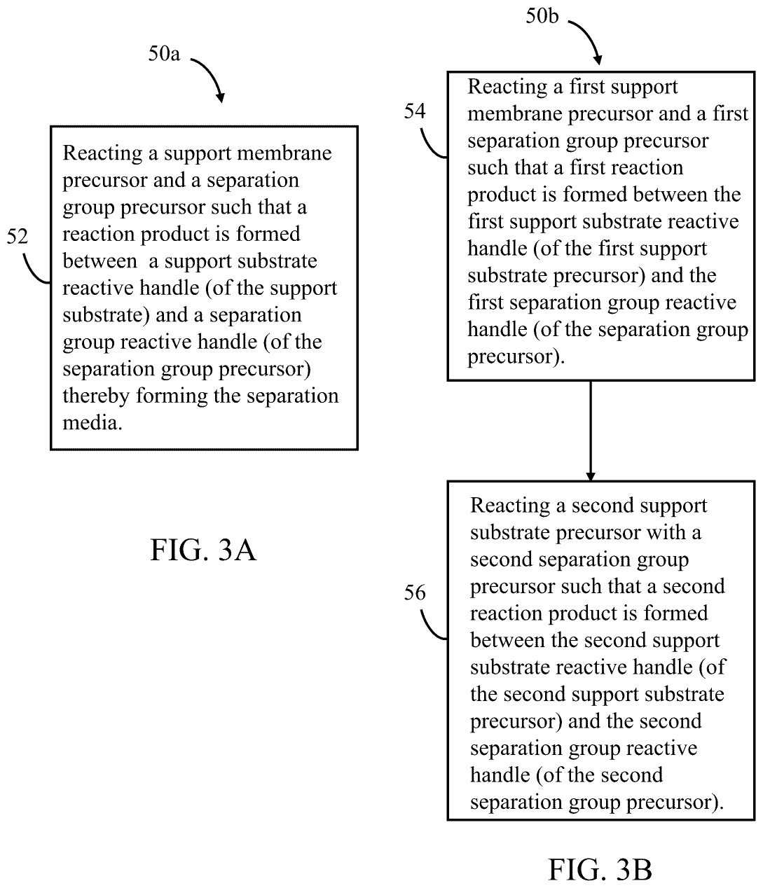

In some embodiments, method 10a or 10b may include method 50a. FIG. 3A is a flow diagram outlining method 50a for making a separation media including a separation ligand of the present disclosure. Method 50a may be understood in reference to synthetic scheme S1 as described herein; however, it is understood that method 50a is not limited to the synthetic scheme S1. The separation ligand of the separation media made from method 50a is synthesized from two components, a separation group precursor (e.g., Pre-Z(1)) and a support substrate precursor (e.g., Pre-M(1)). The separation group precursor includes the separation group (Z) and a separation group reactive handle (Rh¹). The support substrate precursor includes a support substrate (thick vertical black line) and a support substrate reactive handle (Rh²). The separation group reactive handle and the support substrate reactive handle are cooperative handles. Method 50a includes reacting a support substrate precursor and a separation group precursor such that a reaction product (e.g., Rp¹) is formed between the support substrate reactive handle (of the support substrate precursor) and the separation group reactive handle (of the separation group precursor) thereby forming the separation media (e.g., the immobilized separation ligand of Formula SLim).

In some embodiments, step 52 may be accomplished using a reaction mixture. The reaction mixture includes a solvent and the separation group precursor. The reaction mixture may be applied to the support substrate, or the support substrate may be submerged in the reaction mixture. The solvent may include an organic solvent, water, or both. In some embodiments, the solvent is an aqueous buffer that includes one or more salts and/or buffering agents as disclosed herein. The reaction mixture may include additional compounds that facilitate the reaction. For example, the reaction mixture may include an acid, a base, an initiator, a catalyst, or any combination thereof.

In some embodiments where the solvent includes an organic solvent, the reaction step is considered to be “organic assisted” or “organic solvent assisted.” In an organic assisted method, the solvent of the reaction mixture includes water and at least one water-miscible organic solvent. Examples of water-miscible organic solvents include ethanol, acetone, acetonitrile, methanol, propanol (e.g., 2-propanol, 1-propanol), 2-butanol, tetrahydrofuran, dimethylformamide, and dimethyl sulfoxide. The ratio of water to organic solvent in the reaction mixture is such that the reaction mixture is at or near the cloud point of the mixture. The cloud point is the point at which a liquid solution undergoes a liquid-liquid phase separation to from an emulsion or a liquid-solid phase transition to form a stable suspension or a precipitate. The cloud point can be visualized by observing the water-to-organic solvent ratio at which the reaction mixture becomes turbid. Without wishing to be bound by theory, it is thought that including an organic solvent in the reaction mixture such that the reaction mixture is at or near the cloud point increases the conjugation reaction efficiency. The organic solvent molecule can displace water molecules in the separation group precursor thereby increasing interactions between the separation group precursor and the support substrate.

It is possible to define a range of appropriate amounts of organic solvent in the reaction mixture in which the upper boundary is expressed by [V %_{cp}+a(100%−V %_{cp})] and the lower boundary is expressed by [V %_{cp}−bV %_{cp}], where “V %_{cp}” is the percent by volume of the organic solvent in the reaction mixture at the cloud point, “a” is the upper deviation from the cloud point, and “b” is the lower deviation from the cloud point. For the purpose of an example, if the percent by volume of the organic solvent in the ligand solution at the cloud point (V %_{cp}) is 60%, and the upper and lower boundaries are defined by a=0.3 and b=0.5, then the corresponding appropriate amounts of organic solvent in the reaction mixture would range from 30% to 72% organic solvent by volume. In embodiments, the reaction mixture can include an amount of organic solvent in which “a” is about 0.01, 0.02, 0.03, 0.04, 0.05,0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99 and “b” is about 0.01, 0.02, 0.03, 0.04, 0.05, 0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99. In some embodiments, the reaction mixture includes an amount of organic solvent ranging from 70% to 130%, 80% to 120%, 90% to 110%, or 95% to 105% of the volumetric amount of the organic solvent at the cloud point of the reaction mixture.

In some embodiments where the reaction mixture is aqueous and includes one or more salts, the reaction step may be kosmotropic salt assisted. In a kosmotropic salt assisted method, the reaction mixture includes water and at least one kosmotropic salt at a concentration such that the reaction mixture is at or near its cloud point. Examples of kosmotropic salts include sodium phosphate, sodium sulfate, and ammonium sulfate. Without wishing to be bound by theory, it is thought that including a kosmotropic salt in the reaction mixture such that the reaction mixture is at or near the cloud point increases the conjugation reaction efficiency. The salt molecules can disrupt the solvation shell of separation group precursors thereby increasing interactions between the separation group precursor and the support substrate.

In some embodiments, the separation media includes a first plurality of separation ligands immobilized on the support substrate and a second plurality of separation ligands immobilized on the support substrate. In some such embodiments, method 50b may be used to prepare the separation media. FIG. 3B is a flow diagram outlining method 50b for making a separation media that includes multiple pluralities of separation ligands immobilized to the support substrate. The first plurality of separation ligands synthesized according to method 50b are made from two components a first support substrate reactive precursor and a first separation group precursor. The second plurality of separation ligands synthesized according to method 50b are made from two components a second support substrate reactive precursor and a second separation group precursor. The first support substrate precursor includes the first support substrate reactive handle. The second support substrate precursor includes a second support substrate reactive handle. The first support substrate reactive handle and the second support substrate reactive handle may be the same or different. The first separation group precursor includes a separation group and a first separation group reactive handle. The second separation group precursor includes separation group and the second separation group reactive handle. The first support substrate reactive handle and the first separation group reactive handle are cooperative reactive handles. The second support substrate reactive handle and second separation group reactive handle are cooperative handles.

Method 50b includes reacting the first support substrate precursor and the first separation group precursor such that a first reaction product is formed between the first support substrate reactive handle (of the first support substrate precursor) and the first separation group reactive handle (of the first separation group precursor). The method further includes reacting the second support substrate precursor with the second separation group precursor such that a second reaction product is formed between the second support substrate reactive handle (of the second support substrate precursor) and the second separation group reactive handle (of the second separation group precursor).

In some embodiments, the first separation group precursor includes an amine, and the entire method (50b) is amine assisted.

In some embodiments, step 54, step 56, or both are organic solvent assisted or kosmotropic salt assisted. For example, step 54 may be accomplished with a first reaction mixture that includes the first separation group precursor, water, and an organic solvent that is miscible with water or a kosmotropic salt. Step 56 may be accomplished with a second reaction mixture. The second reaction mixture includes the second separation group precursor water and an organic solvent that is miscible with water or at least one kosmotropic salt.

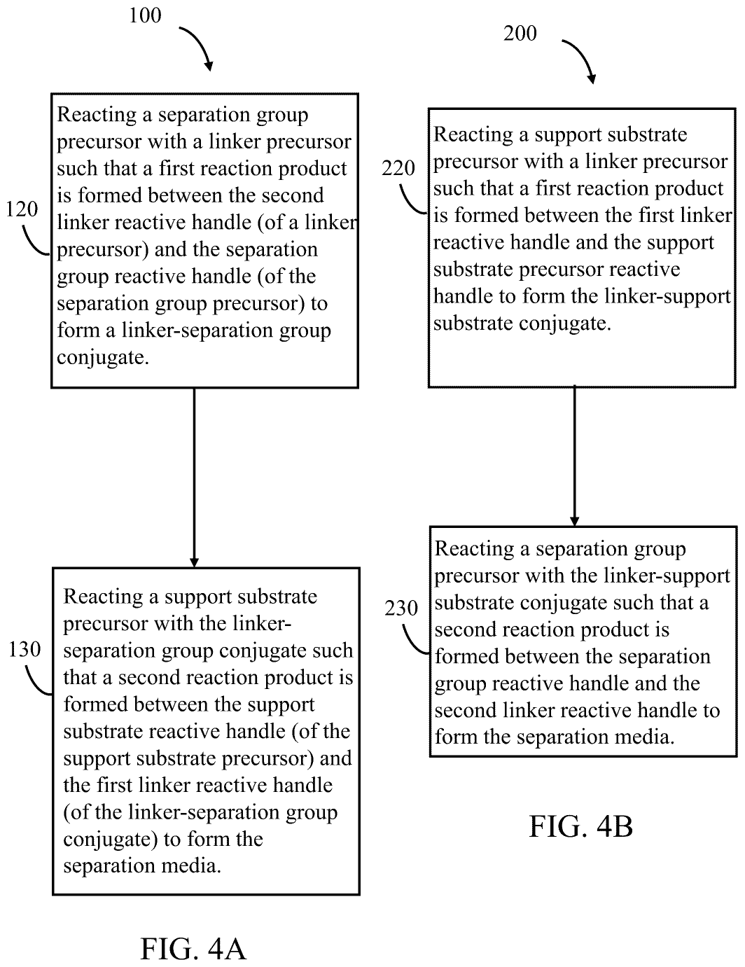

In some embodiments, method 10a, 10b, 50a, or 50b may include method 100. FIG. 4A is a flow diagram outlining method 100. Method 100 may be understood in reference to synthetic scheme S2 as described herein; however, it is understood that the method of 100 is not limited to the synthetic scheme S2. The separation ligands of the separation media made according to method 100 are synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (thick vertical black line) and a support substrate reactive handle (Rh⁵). The second linker reactive handle (Rh 4) and the separation group reactive handle (Rh⁶) are cooperative reactive handles. The first linker reactive handle (Rh³) and the support substrate reactive handle (Rh⁵) are cooperative reactive handles.

The method 100 includes reacting the separation group precursor with the linker precursor such that a first reaction product is formed between the second linker reactive handle (of a linker precursor) and the separation group reactive handle (of the separation group precursor) to form a linker-separation group conjugate (step 120). Method 100 further includes reacting the support substrate precursor with the linker-separation group conjugate of step 120 such that a second reaction product is formed between the support substrate reactive handle (of the support substrate precursor) and the first linker reactive handle (of the linker-separation group conjugate) to form the separation media (step 130).

In some embodiments, method 10a, 10b, 50a, or 50b may include the method 200. FIG. 4B is a flow diagram outline method 200. The method 200 may be understood in reference to synthetic scheme S3 as described herein; however, it is understood that the method of 200 is not limited to the synthetic scheme S3. The separation ligand made according to method 200 is synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (M) and a support substrate reactive handle (Rh⁵). The second linker reactive handle (Rh⁴) and the separation group reactive handle (Rh⁶) are cooperative reactive handles. The first linker reactive handle (Rh³) and the support substrate reactive handle (Rh⁵) are cooperative reactive handles.

Method 200 includes reacting a support substrate precursor with a linker precursor such that a first reaction product is formed between the first linker reactive handle and the support substrate precursor reactive handle to form the linker-support substrate conjugate. Method 200 further includes reacting a separation group precursor with the linker-support substrate conjugate such that a second reaction product is formed between the separation group reactive handle and the second linker reactive handle to form the separation media.

Any step of method 100 or method 200 may be organic solvent assisted or kosmotropic salt assisted.

In some embodiments, methods 10a, 10b, 50a, 50b, 100, and 200 further include functionalizing the support substrate to install the support substrate reactive handles. Installing the support substrate reactive handles followed by one or more conjugation reactions to immobilize the separation ligands to the reactive handles is called indirect immobilization. In such embodiments, the method may further include depositing a polymer having reactive handles onto the support substrate. In some embodiments, the polymer is deposited such that is grafted onto the support substrate. In other embodiments, the polymer is deposited such that it is grafted from the support substrate. In embodiments where the polymer is grafted from the support substrate, the method may further include coupling an initiator to the support substrate to form an immobilized initiator. In such embodiments, the method may further include polymerizing a plurality of monomers from the immobilized initiator.

In some embodiments, the support substrate reactive handle is already a part of the support substrate and not from a deposited functional layer. In such embodiments, the separation ligands are immobilized directly to the support substrate in a process called direct immobilization. Any of the methods 10a, 10b, 50a, 50b,100, and 200 may include direct immobilization.

Direct and indirect immobilization may be accomplished using the amine assisted method, without amine assistance groups (not amine assisted), using the organic solvent assistance method, not using the organic solvent assistance method, using the kosmotropic assisted method, not using the kosmotropic salt assisted method, or any combination thereof.

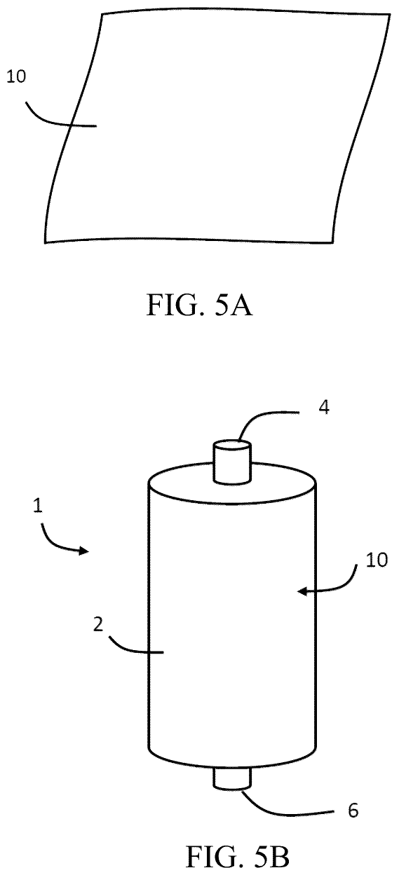

The separation media of the present disclosure may be employed in a separation device. The separation device may be a membrane chromatography column, a membrane chromatography cassette, or other membrane chromatography device that includes the separation media of the present disclosure. A separation device may be operated manually or integrated with software, pumps, detectors, and other accessories. The separation media 10 is schematically shown as a membrane in FIG. 5A. The separation media membrane 10 may be provided in a separation device 1 (e.g., a chromatography column), shown in FIG. 5B. The separation device 1 includes a housing 2 with an inlet 4 and an outlet 6 to facilitate flow through the device.

In some embodiments, two or more separation media of the present disclosure may be arranged in a stacked configuration. The stacked configuration may be employed in a separation device. In some embodiments, a first separation media and a second separation media are arranged in a stacked configuration. In some embodiments, the first separation media and the second separation media have the same identity; that is, the separation media have the same support substrate and the same separation ligands immobilized on the substrate. The separation ligands are immobilized at the same or similar separation ligand densities. In other embodiments, the first separation media and the second separation media have different identities. For example, the first separation media and the second separation media have a different support substrate; different separation ligands; different separation group densities; or any combination thereof.

The separation device (e.g., membrane chromatography column, membrane chromatography cassette, or other membrane chromatography device) may provide a residence time of 5 minutes or less, 2 minutes or less, 1 minute or less, 30 seconds or less, 10 seconds or less, 6 seconds or less, 5 seconds or less, 4 seconds or less, 3 seconds or less, 2 seconds or less, or 1 second or less. The separation device (e.g., membrane chromatography column, membrane chromatography cassette, or other membrane chromatography device) may provide a residence time of 0.01 seconds or greater, 0.1 seconds or greater, 1 second or greater, 5 seconds or greater, 6 seconds or greater, 10 seconds or greater, 30 seconds or greater, 1 minute or greater, or 2 minutes or greater. Residence time is the time any normalized amount of fluid takes to traverse the separation media of the separation device (a single separation media or multiple separation media). For example, residence time is the time it takes any molecule that is not the target and/or does not bind to the separation media to traverse the separation media in a separation device. Residence time is calculated as the flow rate or the solution going through the column divided by the total bed volume of all of the separation media included in the separation device. The residence times of the separation devices of the present disclosure may be lower than those of separation media made of resins.

Process productivity can be defined using the equation below. In the denominator, Vₜₒₜ is the total volume of solution passing through the separation media (e.g., column or cassette) during the whole process, including load (the volume of the isolation solution discussed herein), rinse (e.g., the volume of the washing solution as discussed herein), elution (e.g., the volume of the elution solution as discussed herein), and regeneration steps (e.g., the volume of the regeneration solution as discussed herein). BV is the chromatography medium bed volume (corresponding to the volume of the separation media), and τ (tau) is residence time. Loading volume is proportional to dynamic binding capacity of the chromatography column medium. Thus, process productivity increases with increasing binding capacity and decreasing residence time.

$$Productivity = \frac{target}{Cost of time} = \frac{Loading volume \times target \times yeild}{( \frac{{V}_{tot}}{BV} ) \times τ}$$

Dynamic binding capacity generally refers to the concentration of bound target on the separation media (milligram bound per unit bed volume of separation media) at breakthrough in the effluent. A dynamic binding capacity at 10% breakthrough (DBC_{10%}) can be determined via a standard chromatography method, e.g., using Cytiva ÄKTA pure Fast Protein Liquid chromatography (FPLC). First, the separation media is packed into a housing unit. Then, the contained separation media is connected to an FPLC system. Next, feed material (e.g., isolation solution) containing the target is passed though the separation media under certain column volumes per minute flowrate (CV/min) until the effluent concentration of the target reaches 10% of the feed concentration, as determined by a detector (e.g., a UV detector) . At the end, based on the holdup volume in the FPLC system and separation media volume, the DBC_{10%} is calculated as follows ((Volume to 10% breakthrough-holdup volume)×(feed concentration))/(volume of separation media)=DBC_{10%} expressed as mg target material/unit volume separation media. The volume of the separation media is determined by the surface area of the separation media multiplied by the thickness of the separation media. The volume of the separation media can be referred to as the bed volume. In general, the volume of the separation media does not account for the void space within the separation media. The holdup volume is the total volume between the injection port (i.e., the location where a fluid enters the system) and the detector. The holdup volume includes the bed volume (e.g., the separation media volume) as well as any volume between the injection port and the bed and any volume between the bed and the detector.

In some embodiments, a separation media or separation device containing the same has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media has a dynamic binding capacity at 10% breakthrough of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The dynamic binding capacity may depend at least in part on the target and the affinity group.

The separation media of the present disclosure may have a variety of static binding capacities (SBC). The static binding capacity is the amount of target bound to the separation media per volume of the separation media. The static binding capacity can be determined, for example, by incubating the separation media with an isolation solution containing a known amount of the target ligand for a period of time. Following incubation, the amount of the target still in the isolation solution (target not bound to the separation media) can be measured. The static binding capacity can then be calculated as the difference between the initial amount of the target in the isolation solution and the amount of target in the isolation solution following incubation with the separation media. The amount of the target in the isolation solution pre- and post-incubation with the separation media can be determined, for example, using spectroscopy and/or high performance liquid chromatography.

The static binding capacity may be higher than the dynamic binding capacity at 10% breakthrough. For example, in some embodiments, the SBC can be 10% to 40% greater than the DBC_{10%}. The pore size of the support substrate may influence the SBC and DBC_{10%}. For example, smaller pore sizes may cause a greater difference between the SBC and the DBC_{10%} as compared to relatively larger pore sizes. In some embodiments, a separation media or separation device containing the same has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media or separation device containing the same has a static binding capacity of 150 mg/mL bed volume or less, 120 mg/mL bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The static binding capacity may depend at least in part on the target and the affinity group.

The separation media may have a variety of separation ligand densities. Separation ligand density is the amount of separation ligands immobilized per unit volume of the separation media. In embodiments where the separation media only includes separation groups that include affinity groups, the separation group density can be a measure of affinity group density. The separation ligand density can be determined, for example, by incubating the support substrate (for example, according to 51, S2, or S3) with the reaction solution containing a known amount of the separation group precursor for immobilization for a reaction time to form the separation media. Following incubation, the amount of the separation group precursor containing still in the reaction solution (unreacted) can be measured. The density of the separation ligands can then be calculated as the difference between the initial amount of the separation group precursor in the reaction solution and the amount of separation group precursor in the reaction solution following incubation with the support substrate. The amount of the separation group precursor in the reaction solution pre- and post-incubation with the support substrate can be determined, for example, using spectroscopy and/or high performance liquid chromatography. The separation group pre-cursor can be used as a proxy for the separation ligand.

In some embodiments, the separation media has separation ligand density of 0.01 milligrams of separation ligands per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/mL bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.

Separation ligand density can also be described as the specific surface area (SSA) in square meters (m²) relative to the bed volume of the separation media. SSA can be determined, for example, using nitrogen Brunauer-Emmett-Teller (BET) analysis. Prior to immobilization of the separation ligands on the support substrate, the support substrate will have a support substrate SSA. After immobilization of the separation ligands on the support substrate to form the separation media, the separation media has a separation media SSA. The support substrate SSA and the separation media SSA may be impacted by the pore size of the support substrate, Generally, support substrates with greater pore sizes have a larger support substrate SSA. Generally, the separation media SSA will be greater than the support substrate SSA. In some embodiments the separation media SSA is 0.5 times or greater than the support substrate SSA, 1 time or greater than the support substrate SSA, 1.5 times or greater than the support substrate SSA, 2 times or greater than the support substrate SSA, 3 time or greater than the support substrate SSA, 4 time or greater than the support substrate SSA, 5 time or greater than the support substrate SSA, or 7 time or greater than the support substrate SSA. In some embodiments the separation media SSA is 10 times or less than the support substrate SSA, 7 times or less than the support substrate SSA, 5 times or less than the support substrate SSA, 4 times or less than the support substrate SSA, 3 times or less than the support substrate SSA, 2 times or less than the support substrate SSA, 1.5 times or less than the support substrate SSA, or 1 time or less than the support substrate SSA.

In some embodiments the separation media has a separation SSA of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/ mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/ mL, or 5 m²/mL bed volume to 10 m²/mL when the support substrate has an average pore size of 0.1 μm to 10.0 μm, such as 0.2 μm to 0.5 μm.

In some embodiments, the separation media and/or separation devices containing the same are able to purify a target molecule at a fast flow rate. For example, separation media and/or separation devices containing the same may be used to purify a target at residence times of 5 minutes of less, 2 minutes or less, 1 minute or less, 30 seconds or less, 10 seconds or less, or 6 seconds or less. The residence time is somewhat dependent on the volume of the separation media and/or on the size of the device. For example, in separation media that have low volumes and/or separation devices that are small, the residence times may be as low as 1 second or less. Although there is no desired lower limit for the residence time, in practice residence times are 0.1 seconds or greater.

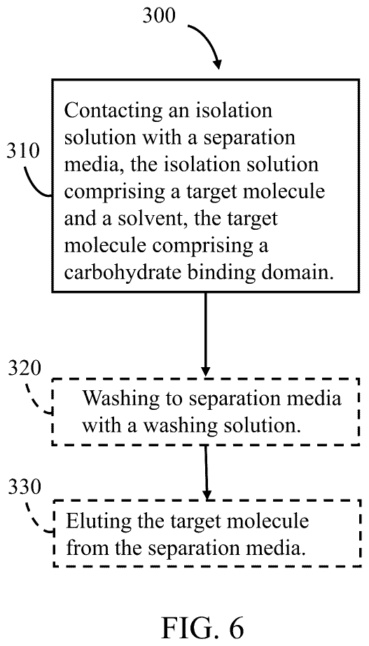

The present disclosure provides methods for using the separation media and/or the separation devices of the present disclosure. FIG. 6 is a flow diagram outlining a method 300 for using the separation media of the present disclosure to isolate and/or concentrate a target molecule from an isolation solution. In some embodiments, the method may be used to remove one or more molecules that have a blood type antigen recognizing domain (e.g., an anti-A blood type antigen antibody, an anti-B blood type antigen antibody, or both) from a blood product. Method 300 includes contacting an isolation solution with a separation media (step 310).

The isolation solution includes a solvent and a plurality of the target molecules. In some embodiments, the isolation solution includes a plurality of target molecules that have already been purified from a mixture that included additional biomolecules. For example, in some embodiments, the isolation solution is a blood product that has had the red blood cells, the white blood cells, platelets, or combinations thereof removed. In some embodiments, the isolation solution is plasma. In some embodiments, the isolation solution may include one or more suitable buffering agents, one or more suitable salts, one or more suitable additives, or any combination thereof.

Examples of suitable salts and buffering agents include sodium chloride; potassium chloride; lithium chloride; rubidium chloride; calcium chloride; magnesium chloride; cesium chloride; tris base (tris(hydroxymethyl)aminomethane); 4-(2-hydroxyethyl)-1-piperazineethanesulfonic acid (HEPES); sodium phosphate; potassium phosphate; ammonium sulfate, 2-(N-morpholino)ethanesulfonic acid (MES); 2,2′,2″-Nitrilotriacetic acid (ADA); N-(2-Acetamido)-2-aminoethanesulfonic acid (ACES); 3-morpholino-2-hydroxypropanesulfonic acid (MOPSO); cholamine chloride hydrochloride; 3-(N-morpholino)propanesulfonic acid (MOPS); N,N-Bis(2-hydroxyethyl)-2-aminoethanesulfonic acid (BES); 2-{[1,3-Dihydroxy-2-(hydroxymethyl)propan-2-yl]amino}ethane-1-sulfonic acid (TES); 3-(N,N-Bis [2-hydroxyethyl]amino)-2-hydroxypropanesulfonic acid (DIPSO); 3-[N-Tris(hydroxymethyl)methylamino]-2-hydroxypropanesulfonic acid (TAPSO); acetamidoglycine; piperazine-1,4 BIS(2-hydroxypropanae sulphonic acid) (POPSO); N-(Hydroxyethyl)piperazine-N′-2-hydroxypropanesulfonic acid (HEPPSO); 3-[4-(2-Hydroxyethyl)piperazin-1-yl]propane-1-sulfonic acid (HEPPS); N-(Tri(hydroxymethyl)methyl)glycine (tricine); 2-Aminoacetamide; glycylglycine; N,N-Bis(2-hydroxyethyl)glycine; N-Tris(hydroxymethyl)methyl-3-aminopropanesulfonic acid (TAPS); and the like. Suitable salts and/or buffering agents may be added in an amount of 1 mM or greater, 5 mM or greater, or 10 mM or greater, 20 mM or greater, 50 mM or greater, 100 mM or greater 200 mM or greater, or 500 mM or greater. Suitable salts may be added in an amount of 1 M or less, 500 mM or less, 100 mM or less, 50 mM or less, or 30 mM or less. The salts may be added in an amount ranging from 1 mM to 1 M, 1 mM to 500 mM, 1 mM to 200 mM, 1 mM to 100 mM, 1 mM to 50 mM, 5 mM to 30 mM, 5 mM to 20 mM, or 20 mM to 100 mM.

In some embodiments, the isolation solution includes one or more kosmotropic salts, one or more chaotropic salts, or both. Kosmotropic salts are known as salts that decrease the solubility of nonpolar substances in aqueous solutions, while chaotropic salts increase their solubility. In some embodiments, the amount and/or identity of a kosmotrope and/or chaotropic salts may be designed to increase the binding affinity and/or binding specificity between the target molecules and the affinity groups and/or assistance groups (if present).

Examples of kosmotropic salts that may be present in the isolation solution include ammonium sulfate, ammonium phosphate, potassium phosphate, sodium sulfate, sodium chloride, and combinations thereof. Suitable kosmotropic salts may be present in the isolation solution in an amount of 0.1 M or greater, 0.5 M or greater, or 1.0 M or greater, or 2.0 M or greater. Suitable kosmotropic salts may be present in the isolation solution in an amount of 6.0 M or less, 5.0 M or less, or 4.0 M or less. The kosmotropic salts may be added in an amount ranging from 0.1 M to 6M, 0.5 M to 2.5 M, or 0.5 M to 3.0 M.

Examples of chaotropic salts that may be present in the solution include sodium chloride, calcium chloride, magnesium chloride and combinations thereof. In some embodiments, the isolation solution includes 1 M or less, 0.5 M or less, or 0.1 M or less of chaotropic salts. In some embodiments, the isolation solution is free or substantially free of chaotropic salts.

Suitable additives include glycerol and other polyols; protease inhibitors; phosphatase inhibitors; cryoprotectants; detergents; chelating agents; reducing agents; and combinations thereof. Suitable additives may be present in the isolation solution in amounts of 0.01 mM or greater, 0.1 mM or greater, 1 mM or greater, 5 mM or greater, 10 mM or greater, or 20 mM or greater. Suitable salts may be added in an amount of 100 mM or less, 50 mM or less, 30 mM or less, 10 mM or less, 5 mM or less, or 1 mM or less. Suitable additives may be present in the isolation solution in amounts ranging from 0.01 mM to 100 mM, 1 mM to 50 mM, 5 mM to 30 mM, 5 mM to 20 mM, 0.01 mM to 5 mM, or 1 mM to 5 mM.

The isolation solution isolation solvent may be any solvent that does not degrade or react with the target molecule or other biomolecules in the isolation solution. In some embodiments, the solvent is water. In some embodiments, the solvent is an organic solvent such as, for example, methanol, ethanol, isopropanol, and acetonitrile, DMSO, DMF, or combinations thereof. In some embodiments, the majority of the solvent is water (e.g., plasma). Alternatively, in some embodiments, the majority of the solvent may be made up of organic solvents. In some embodiments, the solvent is nonaqueous, e.g., consists of organic solvents.

The pH of the isolation solution may be any pH that does not make the target molecule or other biomolecules in the isolation solution unstable or insoluble. Additionally, the pH of the isolation solution should be such that the separation ligands of the separation media are not unstable. The pH of the isolation solution may be controlled to enhance the binding affinity of the target molecules to the affinity groups and/or assistance group (if present).

The isolation solution is contacted with the separation media such that at least a portion of the plurality of the target molecules bind to at least a portion of the separation ligands that include an affinity group and/or an assistance group (if present). Molecules present in the isolation solution that do not include a blood type antigen recognizing domain (i.e., molecules that are not the target molecules), will not bind to the affinity group or will bind to the affinity group with a lesser affinity than the target molecule. Such off-target molecules can be removed in a washing step as discussed elsewhere herein. Through binding to the affinity group, the target molecules are temporarily immobilized on the support membrane.

During step 310, the temperature at which the separation media or device containing the same is exposed to may be adjusted so as to increase or decrease binding efficiency of the target molecule. For example, in some embodiments, exposing the separation media to a temperature of 37° C. during step 310 may allow for the target molecules to bind to the affinity groups faster than they would if the separation media was exposed to a temperature of 4° C. In some embodiments, the temperature is 4° C. to 37° C.

In some embodiments, the method 300 includes washing the separation media with a washing solution (step 320). Washing the separation media with a washing solution includes contacting the separation media with the washing solution. Washing the separation media may allow for any molecules that are not the target molecule to be removed from the separation media. In the washing step, at least a portion of the target molecules remain bound to the affinity groups and temporarily immobilized on the support substrate. In some embodiments, it may be desirable to minimize the volume of washing solution used as to not dilute the molecules of the isolation solution that includes a blood product.

The washing solution may include a variety of components or may simply be a solvent (e.g., water). The composition and/or pH of the washing solution should be such that none of the components degrade or react with the target molecule. Additionally, the composition and/or pH should be such that the washing solution does not decrease the affinity of the target molecule to the affinity group to a point where the target molecule is able to be removed from the affinity group and washed through the separation media. The washing solution includes a washing solvent. The washing solvent may be water, an organic solvent, or both. The washing solvent may be any solvent as described herein such as those described relative to the isolation solution. In embodiments, the washing solution includes one or more buffering agents, one or more salts, one or more additives, or combinations thereof. The one or more salts, one or more buffering agents, or one or more additives may be present in the washing solution in any amount as described relative to the isolation solution.

The pH of the washing solution may be any pH that does not make the target molecule or other biomolecules in the isolation solution unstable or insoluble. Additionally, the pH of the washing solution should be such that the separation ligands of the separation media are not unstable. The pH of the washing solution may be controlled to enhance the binding affinity of the target molecules to the affinity groups and/or decrease the affinity of off target molecules to the affinity groups.

In some embodiments, step 320 may be repeated with additional washing solutions. The additional washing solutions may have the same composition and/or pH as the first washing solution or a different composition and/or pH as the first washing composition.

In some embodiments, method 300 further includes eluting the plurality of target molecules that were temporarily immobilized on the support membrane (step 330). The target molecules are eluted using by contacting the separation media with an elution solution. The elution solution may be of any composition and/or pH that allows for the target molecules to be separated from the affinity groups and exit the separation media. As such, the elution solution generally is of a pH or includes a composition that decreases the affinity of the target molecule for the affinity group or has a higher affinity for the affinity group such as to compete of the target molecule. Additionally, if assistance groups are present, the elution solution generally is of a pH or includes a composition that decrease the electrostatic and/or hydrophobic interactions between the assistance groups and the target molecule.

Methods for eluting the target molecules include using an elution solution that has a higher conductivity and/or salt composition than the washing and/or isolation solution; using an elution solution that has a different pH than the washing and/or isolation solution; using an elution solution that has a different solvent or mixture of solvents than the washing and/or isolation solution; and combinations thereof. The composition of the elution solution may be designed for specific target molecules. Different target molecules (or target molecules and other molecules) may be eluted using a linear gradient elution or using a step isocratic elution.

In some embodiments, the elution solution includes high amounts of one or more salts in order to decrease the binding affinity between the target molecule and the affinity groups and/or assistance groups (if present). The salt or mixture of salts may be any salt as described herein, for example, in reference to the isolation solution. The salt or mixture of salts may be present in the elution solution in an amount of 50 mM or greater, 100 mM or greater, 150 mM or greater, 200 mM or greater, 300 mM or greater, 500 mM or greater, or 1 M or greater. The salt or mixture of salts may be present in the elution solution in an amount of 5 M or less, 1 M or less, 500 mM or less, 300 mM or less, 200 mM or less, or 100 mM or less.

In some embodiments, the amount and/or identity of a kosmotrope and/or chaotropic salts may be designed to decrease the binding affinity between the target molecules and the affinity groups and/or assistance groups (if present).

In other embodiments, the pH of the elution solution may be such as to decrease the binding affinity between the target molecules and the affinity groups. Without wishing to be bound by theory, the pH of the solution may impact the strength and/or availability of various affinity group-target molecule interactions such as hydrogen bonding interactions, electrostatic interactions, hydrophobic interactions, or combinations thereof. In some embodiments, the pH of the elution solution may be higher than the pH of the washing and/or isolation solution. In some embodiments, the pH of the elution solution may be lower than the pH of the washing/isolation solution.

In some embodiments, the elution solution may include a molecule that is bound by the affinity group and/or the target molecule. As such the molecule can compete for binding to the affinity group and/or the target molecule. Such a molecule may be present in an amount such as to compete off the target molecules from the affinity groups. To that end, in some embodiments, the elution solution includes an affinity group competitive molecule and a solvent.

An affinity group competitive molecule is a molecule that binds to the affinity group, and/or the target molecule when present at a sufficient concentration can compete off the target molecule from the affinity group. In some embodiments, the affinity group competitive molecule has a higher affinity for the affinity group than the target molecule. In other embodiments, the affinity group competitive molecule has a lower affinity for the affinity group than the target molecule. In yet other embodiments, the affinity group competitive molecule may have the same affinity for the affinity group as the target molecule. In some embodiments, the affinity group competitive molecule has a higher affinity for target molecule than the affinity group. In other embodiments, the affinity group competitive molecule has a lower affinity for the target molecule than the affinity group. In yet other embodiments, the affinity group competitive molecule may have the same affinity for the target molecule as the affinity group.

An affinity group competitive molecule may be any molecule that binds to a given affinity group and/or a given target molecule. In some embodiments, an affinity group competitive molecule is a monosaccharide, a disaccharide, or a glycan. In some such embodiments, the affinity group competitive molecule may include the same blood type antigen or portion thereof as the affinity group. In some embodiments, the affinity group competitive molecule does not include the same blood type antigen or portion thereof as the affinity group. The affinity group competitive molecule may be chosen based on the identity of the target molecule and/or the affinity group.

An affinity group competitive molecule may be present in an elution solution at a concentration sufficient to compete off the target molecules from the affinity groups. In some embodiments, the affinity group competitive molecule may be present in an elution solution the amount of 20 mM or greater, 50 mM or greater, 100 mM or greater, 200 mM or greater, 300 mM or greater, 400 mM or greater, or 500 mM or greater. In some embodiments, the affinity group competitive molecule may be present in an elution solution the amount of 1 M or less, 500 mM or less, 400 mM or less, 300 mM or less, 200 mM or less, 100 mM or less, or 50 mM or less. In some embodiments, the affinity group competitive molecule may be present in an elution solution the amount of 20 mM to 400 mM, 50 mM to 200 mM, or 100 mM to 500 mM.

In some embodiments, the method 300 includes regenerating the separation media. Regeneration is done to prepare the separation media (or the separation media of a separation device) for subsequent uses. Regeneration may include washing the separation media with a solution designed to strip any molecule that is not covalently attached to the support membrane form the separation media. Regeneration may also include flowing an equilibration solution through the separation media such as to prepare the separation media for future use. In some embodiments, the equilibrium solution may be the same as the isolation solution but without the target molecule or the same as the washing solution.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

### Exemplary Embodiments

The following is a non-limiting list of exemplary embodiments according to the present disclosure.

Embodiment 1 is separation media that includes a support substrate and a plenarily of separation ligands of the formula SL immobilized on the support substrate. The formula SL is

-L-Z (SL)

, where L is a linker and Z is a separation group. The separation group includes an affinity group. The affinity group includes a blood type antigen or a fragment thereof. In some embodiments, the separation media is configured for isolating a target molecule, the target molecule including a blood type antigen recognizing domain. The target molecule may be isolated from an isolation solution that includes the target molecule.

Embodiment 2 is the separation media of embodiment 1, wherein SL is or formula SL1 or SL2.

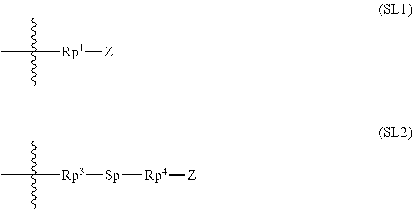

In formula SL1 and SL2 Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof wherein Rp^{A}, Rp^{B}, Rp^{C}, Rp^{D}, Rp^{E}, Rp^{F}, Rp^{G}, Rp^{H}, Rp^{I}, Rp^{J}, and Rp^{K} are represented by:

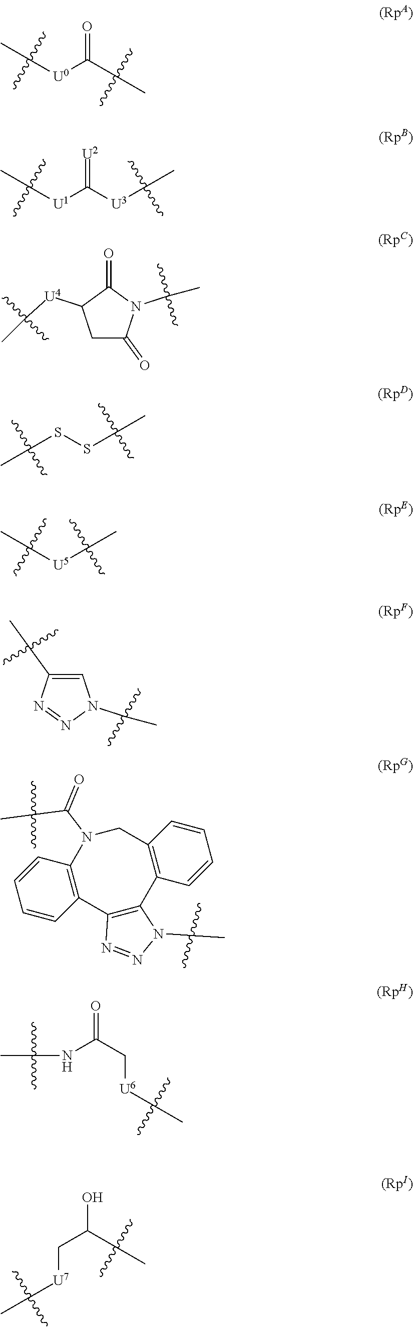

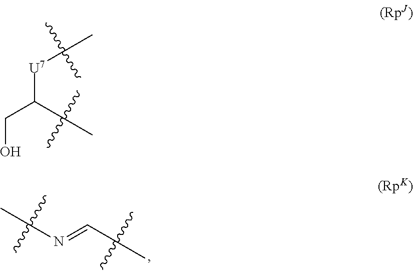

wherein U⁰, U¹, U², U³, U⁴, U⁵, U⁶, and U⁷ are each independently NH, N, O, or S. Sp is a spacer.

Embodiment 3 is the separation media of embodiment 1 or 2, where U⁰, U¹, U², U³, U⁴, U⁵, U⁶, and U⁷ are each independently NH, O, or S.

Embodiment 4 is the separation media of any one of embodiments 1 to 3, wherein the plurality of separation ligands are of formula SL2 and Sp is an alkanediyl or alkenediyl comprising one or more catenated functional groups. In some embodiments, the alkanediyl or alkenediyl comprises a backbone chain of length C1 to C18.

Embodiment 5 is the separation media of embodiment 4, where the alkanediyl or alkenediyl comprises a backbone chain of length C1 to C3.

Embodiment 6 is the separation media of any one of embodiment 1 to 5, where the spacer includes —C(O)—.

Embodiment 7 is the separation media of any one of embodiment 1 to 6, where wherein Rp³, Rp⁴, or both comprises Rp^{E}.

Embodiment 8 is the separation media of any one of embodiment 1 to 6, wherein Rp³ and Rp⁴ comprises Rp^{E}.

Embodiment 9 is the separation media of embodiment 8, where each U⁵ is O.

Embodiment 10 is the separation media of embodiment 8, where each U⁵ is NH.

Embodiment 11 is the separation media of embodiment 8, where one U⁵ is NH and U⁵ is O. In some embodiments, the U⁵ of Rp³ is O and the U⁵ or Rp⁴ is NH. In some embodiments, the U⁵ of Rp³ is NH and the U⁵ or Rp⁴ is O.

Embodiment 12 is the separation media of embodiment 1 or embodiment 2, where SL2 comprises

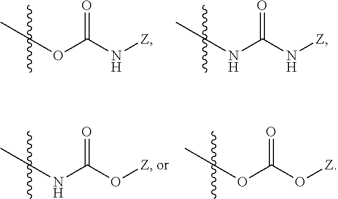

Embodiment 13 is the separation media of embodiment 1 or embodiment 2, where SL2 comprises

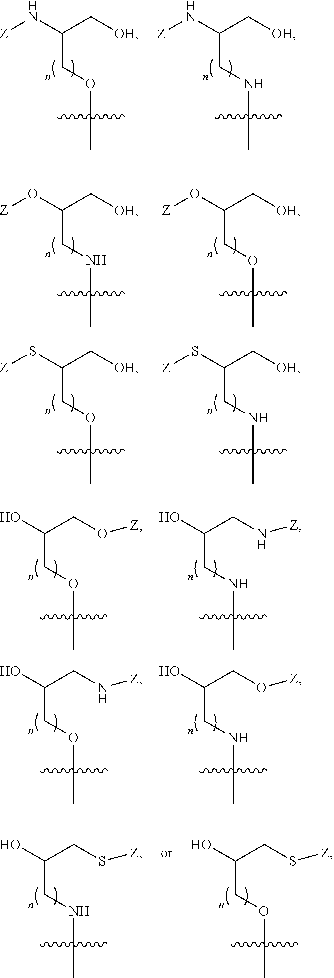

wherein n is 0, 1, 2, 3, 4, 5, 6, 7, 8, 9, or 10. In some embodiments, n is 1.

Embodiment 14 is the separation media of embodiment 1, where the separation ligand formula SL or SL1 is of formula X, XI, XII, or XIII:

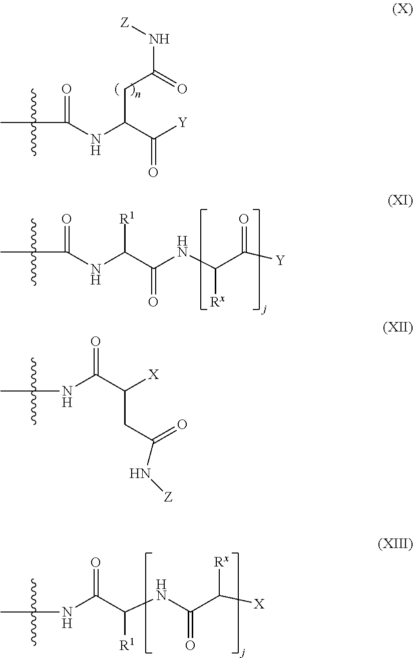

where n is 0, 1, 2, 3, or 4; X is NH₂ or PG_{N} where PG_{N} is an amine protecting group; is Y is OH or a PG_{C(O)OH} where PG_{C(O)OH} is a carboxylic acid protecting group; R¹ is an amino acid side chain; j is 1, 2, 3, 4, 5, 6, 7, 8, 9, or 10; each R^{X} is independently an amino acid side chain or

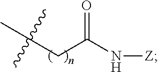

and at least one R^{X} is

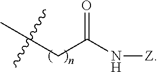

Embodiment 15 is the separation media of any one of embodiments 1 to 14, where the support substrate comprises a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof. In some embodiments, the support substrate includes regenerated cellulose membrane.

Embodiment 16 is the separation media of any one of embodiments 1 to 15, where the separation media is configured for use with an organic solvent.

Embodiment 17 is the separation media of any one of embodiments 1 to 16, where the separation media is configured for use with an aqueous solvent.

Embodiment 18 is the separation media of any one of embodiments 1 to 17, where the blood type antigen or fragment thereof comprises a glycan.

Embodiment 19 is the separation media of any one of embodiments 1 to 18, where the blood type antigen of fragment thereof comprises an A-type antigen, a B-type antigen, or a fragment thereof. In some embodiments, the separation media includes Gal, Fuc, and GalNAc. In some such embodiment, the Gal, Fuc, and GalNAc are arranged in an A-type antigen configuration. In some embodiments, the separation media includes Gal, Fuc, and GlcNAc. In some such embodiments, Gal, Fuc, and GlcNAc are arranged in a B-type antigen configuration. In some embodiments, the blood type antigen includes Glc, Fuc, GlcNAc. In some such embodiments, the Glc, Fuc, GlcNAc are arranged in the H-type antigen configuration.

Embodiment 20 is the separation media of any one of embodiments 1 to 19, where the blood type antigen or fragment thereof comprises the three terminal saccharide residues of the A-type antigen or the B-type antigen.

Embodiment 21 is the separation media of any one of embodiments 1 to 19, where a blood type antibody comprises the blood type recognizing domain.

Embodiment 22 is the separation media of any one of embodiments 1 to 21, where the blood type antibody is the anti-A antibody or the anti-B antibody.

Embodiment 23 is the separation media of any one of embodiments 1 to 22, where the blood type antigen or fragment thereof comprises a first blood type antigen or fragment thereof, wherein the separation media further comprises a second plurality of separation ligands immobilized on the support membrane, the second plurality of separation ligands comprising a second affinity group comprising a second blood type antigen or fragment thereof.

Embodiment 24 is the separation media of embodiment 23, where the first blood type antigen comprises an A-type antigen or fragment thereof and the second blood type antigen comprises a B-type antigen or fragment thereof.

Embodiment 25 is a separation media comprising two or more the separation media of any one of embodiment 1 to 24 arranged in a stacked configuration.

Embodiment 26 is the separation media of embodiment 25, where the separation media comprises two separation media and the separation media are of the same identity.

Embodiment 27 is the separation media of embodiment 25, where the separation media comprises two separation media and the separation media are of a different identity.

Embodiment 28 is a separation device comprising a housing and the separation media of any one of embodiment 1 to 27 disposed within the housing.

Embodiment 29 is a method of removing a target molecule from an isolation solution. The isolation solution includes a blood product and the target molecule. The target molecule includes a blood antigen recognizing domain. The method includes contacting the isolation solution with the separation media of any one embodiment 1 to 27 or the separation device of embodiment 28. The isolation solution is contacted such that at least a portion of the target molecules bind to the separation media.

Embodiment 30 is the method of embodiment 29, where the method further comprises washing the separation media with a washing solution.

Embodiment 31 is the method of embodiment 29 or 30, where the method further comprises eluting the target molecule from the separation media.

Embodiment 32 is the method of any one of embodiments 29 to 31, where the isolation solution comprises a first target molecule and a second target molecule, the first target molecule comprising a first blood type antigen recognizing domain and the second target molecule comprising a second blood type antigen recognizing domain; and the separation media comprises a first blood type antigen capable of binding the first blood type antigen recognizing domain and a second blood type antigen capable of binding the second blood type antigen recognizing domain.

Embodiment 33 is a method of any one of embodiments 29 to 32, where the isolation solution comprises a blood product that does not include red blood cells, white blood cells, platelets, or any combination thereof.

Embodiment 34 is the separation media, separation device, or method of any one of embodiments 1 to 33, wherein the separation media has a static binding capacity of has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, the separation media or separation device containing the same has a static binding capacity of 150 mg/mL bed volume or less, 120 mg/mL bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, the separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.

Embodiment 35 is the separation media, separation device, or method of any one of embodiments 1 to 34, wherein the separation media has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, the separation media has a dynamic binding capacity at 10% breakthrough of 150 mg/mL bed volume or less, 120 mg/mL bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, the separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.

Embodiment 36 is the separation media, separation device, or method of any one of embodiments 1 to 35, wherein the separation media has a separation ligand density of 0.01 milligrams of separation ligands per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/mL bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.

Embodiment 37 is the separation media, separation device, or method of any one of embodiments 1 to 36, wherein the separation media has a separation specific surface area (SSA) of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/mL, or 5 m²/mL bed volume to 10 m²/mL when the support substrate has an average pore size of 0.1 μm to 10.0 μm, such as 0.2 μm to 0.5 μm.
Embodiment 38 is the separation media, separation device, or method of any one of embodiments 1 to 37, wherein the separation media has an average pore size of 10 micrometer (μm) or less, 5 μm or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

### EXAMPLES

These examples are merely for illustrative purposes only and are not meant to be limiting on the scope of the appended claims. All parts, percentages, ratios, etc. in the examples and the rest of the specification are by weight, unless noted otherwise.

### Example 1: Example Synthetic Methods to Prepare the Separation Media of the Present Disclosure

Several synthetic strategies may be employed to construct the separation media of the present disclosure. The synthetic strategies include direct or indirect immobilization of separation ligands. The synthetic strategies also include amine assisted coupling or organic solvent assisted coupling.

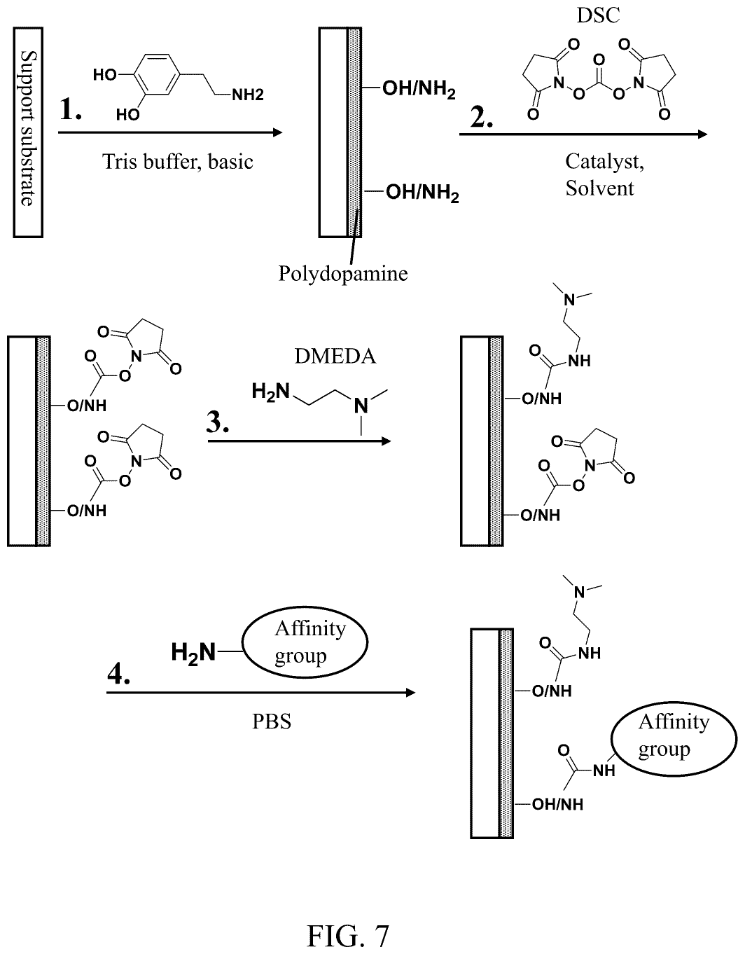

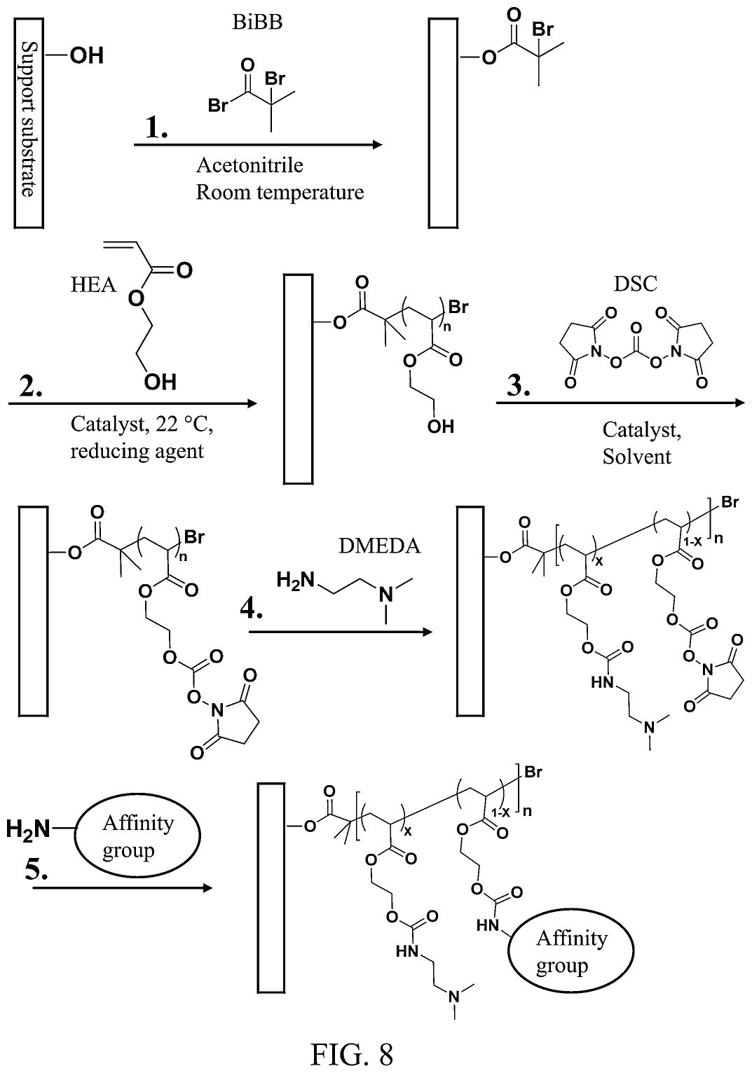

FIG. 7 and FIG. 8 show schematics of synthetic schemes that include the indirect immobilization of the separation ligands to the support substrate. The strategies of FIG. 7 and FIG. 8 also include the amine assisted coupling method.

In Step 1, of the scheme in FIG. 7, polydopamine (PDA) is incorporated onto a support membrane through oxidative polymerization of dopamine in basic aqueous buffer in the presence of air. Deposition of PDA may function to hydrophilize the support substrate and introduce support substrate reactive handles (e.g., hydroxyl, amine, and quinone). In step 2, the PDA reactive handles may be reacted with the bifunctional (has two NHS ester reactive handles) of linker precursor disuccinimidyl carbonate (DSC). The PDA reactive handles (OH and NH₂) may react with the first N-hydroxy succinimidyl (NETS) ester of the linker precursor to form a first amide or carbamate reaction product. In step 3, a portion of the second NHS ester groups may be reacted with the amine reactive handle of N,N-dimethylethylenediamine (DMEDA; a first separation group precursor) to form a second amide reaction product. The DMEDA groups (e.g., the tertiary amine of DMEDA) may act as cationic assistance affinity groups to increase the local concentration of negatively charged (in aqueous buffer) affinity groups at the membrane surface through coulombic interaction. In step 4, the second portion of the second NHS ester groups may be reacted with a second separation group (including an affinity group) precursor reactive handle (an amine as shown in this scheme) to form an amide reaction product. The conjugation reaction of step 4 may be done in an aqueous buffer.

In the first step of the scheme depicted in FIG. 8, an initiator (α-bromoisobutyryl bromide, BiBB) may be coupled to the OH groups of a support substrate. In the second step, hydroxyethyl acrylate monomers may be polymerized from the immobilized initiator to from poly(HEA). In step 3, the poly(HEA) reactive handles (OH groups) may be reacted with the bifunctional (has two NHS ester reactive handles) linker precursor disuccinimidyl carbonate (DSC). The poly(HEA) reactive handles (OH) may react with the first NHS ester of the linker precursor to form a first carbamate reaction product. In step 4, a portion of the second NHS ester groups may be reacted with the amine reactive handle of N,N-dimethylethylenediamine (DMEDA; a first separation group precursor) to form an amide reaction product and install the separation ligands containing the amine assistance groups. The DMEDA groups (e.g., the tertiary amine of DMEDA) may act as cationic assistance groups which may allow for a higher density of negatively charged (in aqueous buffer) affinity groups at the support substrate surface through coulombic interaction. In step 5, the second portion of the second NHS ester groups may be reacted with a second separation group (including the affinity group) precursor reactive handle to form an amide reaction product and install the separation ligands containing the affinity group on the support substrate. The conjugation reaction of step 5 may be completed in an aqueous buffer reaction mixture. In some embodiments, the support substrate may be exposed to a tris base solution as a final step to quench unreacted NHS intermediates and to install separation ligands containing a capping group.

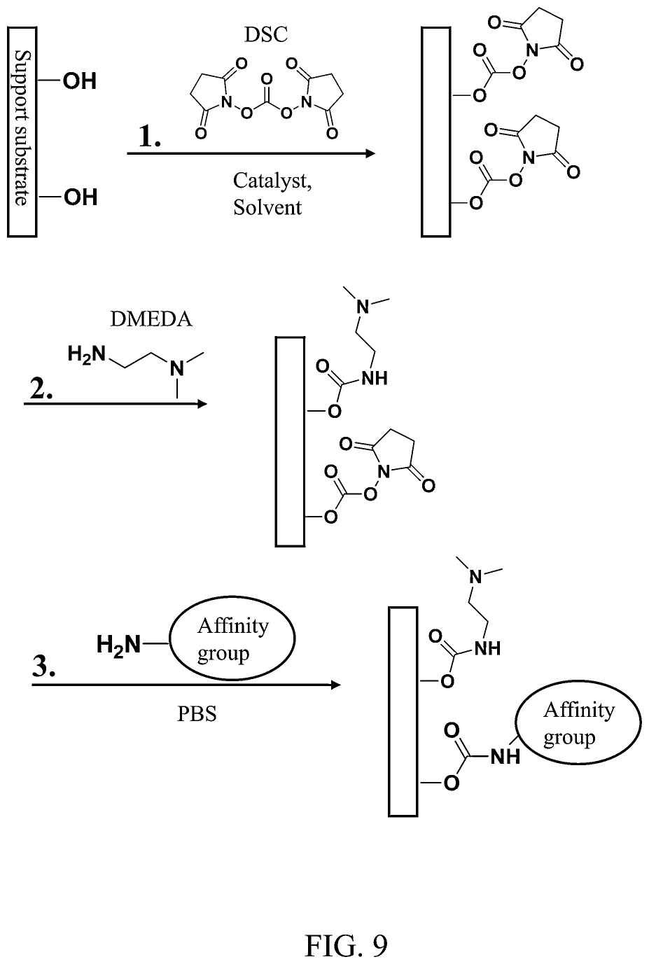

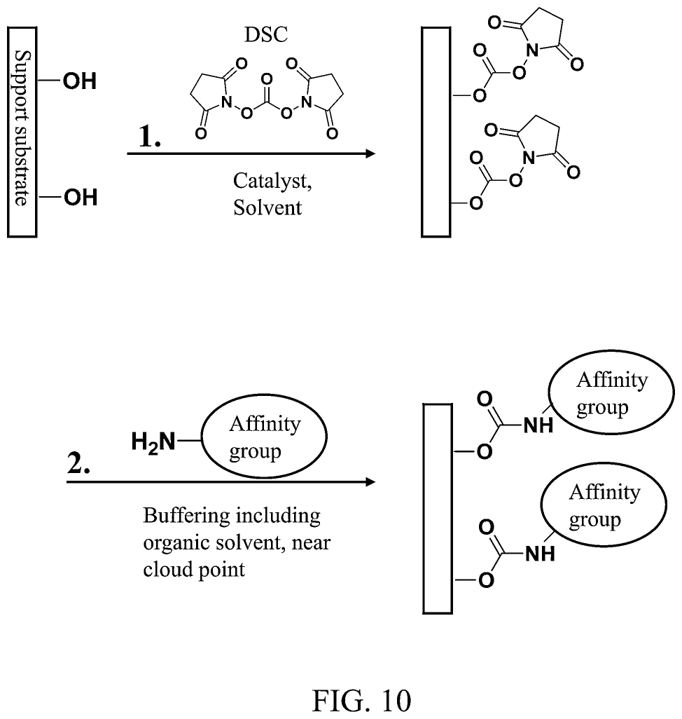

FIG. 9 and FIG. 10 show schematics of synthetic schemes that include the direct immobilization of the separation ligands on the support substrate. The strategy of FIG. 9 includes the amin assisted cooling method. The strategy of FIG. 10 includes the organic solvent assistance method.

FIG. 9 shows a synthetic strategy where the separation ligands can be directly immobilized on the support substrate and the amine assisted method may be used to achieve a high density of separation ligands. This synthetic strategy is similar to the strategy in FIG. 7 except that the membrane was not functionalized with a polymer. Instead, the hydroxyl reactive handles of the support substrate can be directly reacted with one of the NHS ester reactive handles of DSC to form a carbamate reaction product (step 1). Separation ligands having an amine assistance group can be installed (step 2). The amine assistance group may facilitate the installation of the separation groups that include an affinity group (step 3). The conjugation reaction of step 3 may be done in an aqueous buffer reaction mixture.

FIG. 10 shows a synthetic strategy where the separation ligands can be directly immobilized on the support substrate and the organic solvent assisted method may be used to achieve a high density of separation ligands. Residual tertiary amine moieties in the final separation media may have the potential for nonspecific binding when the solution conductivity is very low. As affinity chromatography typically is performed at conductivity levels above that which tertiary amines retain significant binding capacity, the residual amine groups were expected to have negligible effect on chromatographic performance. In an effort to circumvent this potential issue completely, an organic solvent assisted coupling method may be employed to install the separation ligands containing the affinity group. The organic assisted coupling method utilizes water-miscible organic solvents as a constituent of the coupling solution to increase separation group precursor coupling efficiency, which enables use of low separation group precursor concentrations in the coupling solution. Additions of organic solvents to the aqueous buffered reaction mixture (10%-80% by volume depending on the organic solvent used) to bring solution near the cloud point. At the cloud point, the reaction mixture starts to appear turbid upon increasing the concentration of organic solvent. Organic solutions replace water molecules in the separation group precursor solvation shell which can facilitate greater interaction between the separation group precursor and the support substrate. Additional organic solutions added beyond the cloud point may exacerbate aggregation and flocculation dynamics of the separation group precursor, which can comparatively reduce efficiency of coupling reaction. This coupling methodology may allow for high performance separation media to be prepared with low separation group consumption.

FIG. 10 shows an example synthetic scheme that may be used to prepare separation media of the present application via direct immobilization of the separation ligands using the organic solvent assisted coupling method. In step 1 the support substrate reactive handles (OH) may be reacted with the first N-hydroxy succinimidyl (NHS) ester of the DSC linker precursor to form a carbamate reaction product. In the second step, a reaction mixture that is near the cloud point that includes the separation group precursor (includes the affinity group), water, and a water-miscible solvent may be exposed to the reaction product of step 2. The second NHS ester groups of the support substrate-linker conjugate may react with the reactive handle (NH₂) of the separation group precursor to form an amide reaction product and install the separation ligands having the affinity group on the support substrate.

### Example 2: Assessment of Separation Media for Binding Mice IgM Anti-A and Anti-B Antibodies as a Proof of Concept

Twelve separation medias were prepared according to two synthetic strategies. The separation medias were tested for the ability to capture mouse anti-A and anti-B IgM antibodies (available from Abcam in Waltham, MA) from an isolation solution at various binding temperatures (i.e., the temperature at which the separation media was exposed to during the incubation of the isolation solution).

A mouse IgM sandwich ELISA assay (available from Abcam, MA) was used to quantify the concentration of a group A antigen antibody (anti-A antibody) prior to incubation with the separation media. IgM stock supernatant was a nominal 1-3 mg/mL, however, quantitation via ELISA registers actual concentration as 0.234 mg/mL. After six times dilution, the feed concentration of the anti-A IgM antibody incubated with the separation media was 0.039 mg/mL. The stock solution was incubated with two separation medias that were made via two different synthetic schemes at either 4° C. or 37° C. for 2 hours. The static binding capacity results are shown in Table 2. The binding of the anti-A IgM antibody to the separation media was more substantial than the separation media control that has not immobilized separation ligands. At least at a 2 hour incubation time, increased temperature does increase binding capacity which corroborates literature as it has been shown 4° C. vs 37° C. can result in over 20 times the time to reach binding equilibrium.

TABLE 2
SBC of various separation media after incubation
with a solution containing anti-A IgM antibodies

| Separation Media | SBC |
|---|---|
| Unmodified Control | 0.26 |
| Separation media made via method 1; incubation temperature of | 2.82 |
| 4° C. | |
| Separation media made via method 2; incubation temperature of | 2.73 |
| 4° C. | |
| Separation media made via method 2; incubation temperature of | 3.54 |
| 37° C. | |

A mouse IgM sandwich ELISA assay (available from Abcam, MA) was used to quantify the concentration of a group B antigen antibody (anti-B antibody) prior to incubation with the separation media. IgM stock supernatant was a nominal 1 1-3 mg/mL, however, quantitation via ELISA registers actual concentration as 0.126 mg/mL. After two times dilution, the feed concentration used was 0.063 mg/mL. The stock solution was incubated with two separation medias that were made via two different synthetic schemes at either 4° C. or 37° C. for 2 hours. The static binding capacity results are shown in Table 3. At least at a 2 hour incubation time, increased temperature does increase binding capacity.

TABLE 3
SBC of various separation media after incubation
with a solution containing anti-B IgM antibodies

| Separation Media | SBC |
|---|---|
| Separation media made via method 1; incubation temperature of | 1.25 |
| 4° C. | |
| Separation media made via method 2; incubation temperature of | 0.47 |
| 4° C. | |
| Separation media made via method 2; incubation temperature of | 1.35 |
| 37° C. | |

### Example 3: Isoagglutinin Removal Process Modeling

Modeling was done to determine the total time needed to remove 1 kg of IgG from an isolation solution using columns of the separation media of the present disclosure and commercially available resin columns (ESHMUNO P resin, available from Millipore in Billerica, MA). The model assumed an IgG concentration of 15 mg/mL in the feed. Based on the manufacturer's notes, ESHMUNO P resins can process 3 kg of IgG per L resin with an assumed residence time of 6 minutes. The performance of the columns of the separation media of the present disclosure were based on the IgM SBS found in Example 2 with an assumed residence time of 6 seconds. Table 4 shows the modeling results.

TABLE 4
Isoagglutinin removal process modeling results

| | Column Size | | Number of | Purification time | |
|---|---|---|---|---|---|
| Column | in Liters | | Cycles | (total) | |
| ESHMUNO P Resin | 0.4 | L | 1 | 17 | hours |
| Column | | | | | |
| ESHMUNO P Resin | 4 | L | 1 | 4 | hours |
| Column | | | | | |
| Separation media column of | 1.2 | L | 1 | 6 | minutes |
| present disclosure | | | | | |
| Separation media column of | 0.4 | L | 3 | 18 | minutes |
| present disclosure | | | | | |

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1-33. (canceled)

34. A separation media comprising:
a support substrate; and
a plurality of separation ligands for formula SL1 or SL2 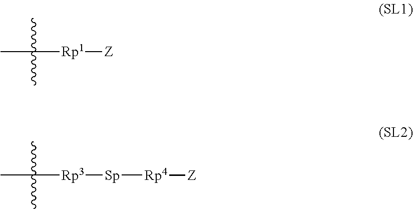
wherein:
Z is a separation group comprising an affinity group, the affinity group comprising a blood type antigen or a fragment thereof; and
Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof, wherein rp^{A}, Rp^{B}, Rp^{C}, Rp^{D}, Rp^{E}, Rp^{F}, Rp^{G}, Rp^{H}, Rp^{I}, Rp^{J}, and Rp^{K} are represented by: 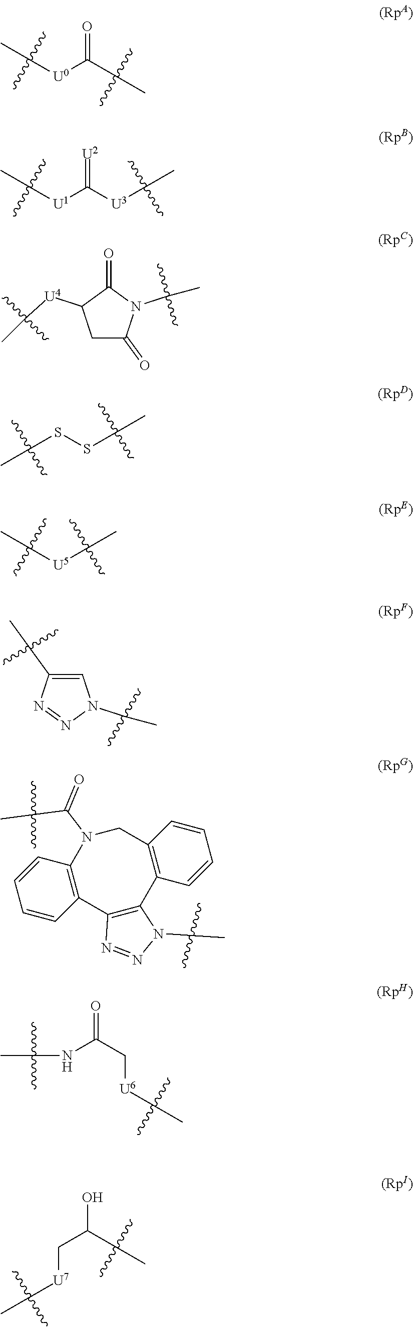 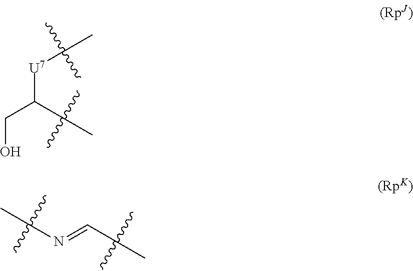
wherein:
U⁰, U¹, U², U³, U⁴, U⁵, U⁶, and U⁷ are each independently NH, O, or S; and
Sp is a spacer comprising a divalent organic group, and
wherein the separation media configured for isolating a target molecule comprising a blood type antigen recognizing domain.

35. The separation media of claim 34, wherein the alkanediyl or alkenediyl comprises a backbone chain of length C1 to C3.

36. The separation media of claim 1, wherein Sp comprises —C(O)—.

37. The separation media of claim 34, wherein Rp³ and Rp⁴ comprises Rp^{E}.

38. The separation media of claim 37, wherein each U⁵ is O, each U⁵ is NH, or one U⁵ is NH and U⁵ is O.

39. The separation media of any one of claim 34, wherein SL2 comprises 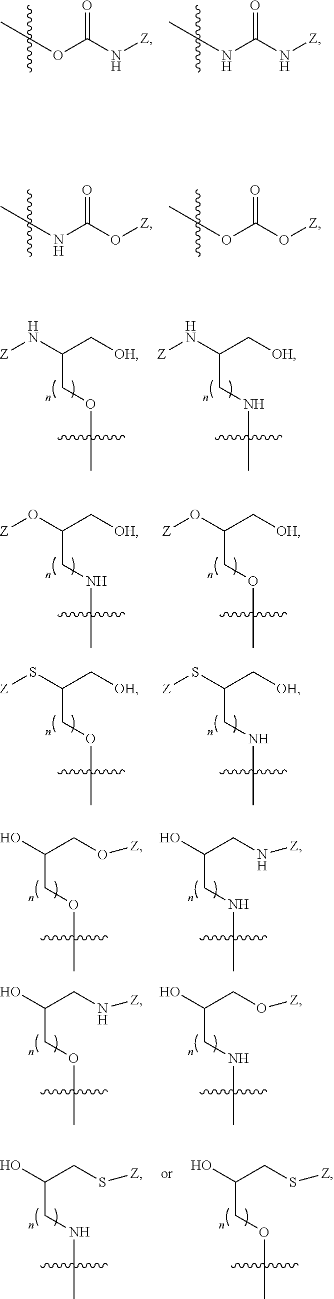
wherein n is 0, 1, 2, 3, 4, 5, 6, 7, 8, 9, or 10.

40. The separation media of claim 34, wherein the separation ligand formula SL or SL1 is of formula X, XI, XII, or XIII: 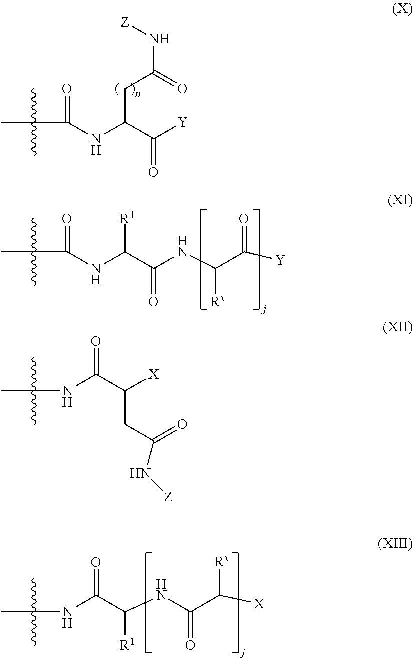
wherein n is 0, 1, 2, 3, or 4;
wherein X in the Formula is NH₂ or PG_{N} where PG_{N} is an amine protecting group;
wherein Y is Y is OH or a PG_{C(O)OH} where PG_{C(O)OH} is a carboxylic acid protecting group;
wherein R¹ is an amino acid side chain;
wherein j is 1, 2, 3, 4, 5, 6, 7, 8, 9, or 10;
wherein each R^{X} is independently an amino acid side chain or 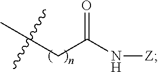
and
wherein at least one R^{X} is

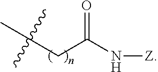

41. The separation media of claim 34, wherein the support substrate comprises a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof.

42. The separation media of claim 34, wherein the blood type antigen of fragment thereof comprises an A-type antigen, a B-type antigen, or a fragment thereof.

43. The separation media of claim 42, wherein the blood type antigen or fragment thereof comprises the three terminal saccharide residues of the A-type antigen or the B-type antigen.

44. The separation media of claim 34, wherein a blood type antibody comprises the blood type recognizing domain and wherein the bloody type antibody is an anti-A antibody or an anti-B antibody.

45. The separation media of any one of claims 34, wherein the blood type antigen or fragment thereof comprises a first blood type antigen or fragment thereof, wherein the separation media further comprises a second plurality of separation ligands immobilized on the support membrane, the second plurality of separation ligands comprising a second affinity group comprising a second blood type antigen or fragment thereof.

46. A separation media comprising two or more of the separation media of claim 34 arranged in a stacked configuration.

47. The separation media of claim 46, wherein the separation media comprises two separation media and the two separation media are of the same identity.

48. The separation media of claim 46, wherein the separation media comprises two separation media and the two separation media are of a different identity.

49. A separation device comprising a housing and the separation media of claim 34 disposed within the housing.

50. A method of removing a target molecule from an isolation solution,
the isolation solution comprising:
a blood product; and
the target molecule comprising a blood antigen recognizing domain;
the method comprising:
contacting the isolation solution with the separation media of claim 34.

51. The method of claim 50, wherein the method further comprises washing the separation media with a washing solution.

52. The method of claim 50, wherein the method further comprises eluting the target molecule from the separation media.

53. The method of claim 50, wherein the isolation solution comprises a first target molecule and a second target molecule, the first target molecule comprising a first blood type antigen recognizing domain and the second target molecule comprising a second blood type antigen recognizing domain; and the separation media comprises a first blood type antigen capable of binding the first blood type antigen recognizing domain and a second blood type antigen capable of binding the second blood type antigen recognizing domain.
